(19) United States
(12) Patent Application Publication
(10) Pub. No.: US 2025/0367110 A1
(43) Pub. Date: Dec. 4, 2025

(54) COMPOSITIONS AND METHODS FOR DELIVERY OF OCULAR THERAPEUTICS

(71) Applicant: The Board of Trustees of the Leland Stanford Junior University, Stanford, CA (US)

(72) Inventors: Eric Andrew Appel, Palo Alto, CA (US); Emily Louise Meany, Palo Alto, CA (US)

(73) Assignee: The Board of Trustees of the Leland Stanford Junior University, Stanford, CA (US)

(21) Appl. No.: 18/830,365

(22) Filed: Sep. 10, 2024

Related U.S. Application Data
(60) Provisional application No. 63/581,931, filed on Sep. 11, 2023.

(51) Int. Cl. A61K 9/00; A61K 31/165; A61K 31/5575; A61K 47/38

(52) U.S. Cl. CPC A61K 9/0024; A61K 9/0051; A61K 31/165; A61K 31/5575; A61K 47/38

(57) ABSTRACT

Compositions for treating ocular disease are disclosed herein. In some embodiments, the composition comprises a dynamic hydrogel comprising a polymer and a plurality of nanoparticles, wherein the polymer is non-covalently crosslinked with the plurality of nanoparticles. The dynamic hydrogel can also comprise an ocular therapeutic encapsulated by the dynamic hydrogel.

### CROSS-REFERENCES TO RELATED APPLICATIONS

This application claims the benefit of U.S. Provisional Patent Application No. 63/581,931, filed on Sep. 11, 2023, which is incorporated by reference in its entirety for all purposes.

### TECHNICAL FIELD

The present technology generally relates to drug delivery, and in particular, to compositions and methods for delivery of therapeutic agents to the eye.

### BACKGROUND

Vision impairment, resulting from eye diseases such as macular degeneration and diabetic macular edema, poses an immense global financial burden and tremendously impacts patients' quality of life. The World Health Organization projects a steady increase in the prevalence of chronic eye diseases over the next ten years, including a 30% increase from 76 to 95.4 million persons with glaucoma and a 20% increase from 195.6 to 243.3 million persons with age-related macular degeneration. The current standard of care utilizes intravitreal (ITV) administration to treat several ocular diseases, with ITV being one of the most effective methods for delivering therapies to the retina. To date, there are various approved ITV biologic therapies, including pegaptanib sodium, ranibizumab, aflibercept, brolucizumab, and more recently faricimab. Yet, despite these robust medical breakthroughs for the management of acquired retinal diseases, patient compliance with repeated ITV injection dramatically falls over time and remains a major obstacle to life-long treatment. Several intervention strategies seek to address this obstacle, including the use of long-acting delivery (LAD) technologies to sustain drug exposure, effectively prolonging efficacy and reducing the frequency of injections. By far, the most advanced LAD technology to successfully address patient compliance is the recently FDA-approved drug, Susvimo™ (Genentech, California, USA), a refillable port delivery system for ranibizumab. This device is surgically implanted at the pars plana and slowly releases ranibizumab into the vitreous humor (VH) of the eye, with a minimum of 24 weeks between refill exchanges. Other approved LAD technologies for ocular use are predominantly biodegradable implants for sustained immunosuppressive steroid delivery, wherein the steroid itself may mitigate any potential immune response to the delivery vehicle. In the face of continuing growth in populations impacted by ocular diseases, advancement of novel targets for the management of these diseases, and the expanding diversity of drug modalities (such as new classes of molecules beyond traditional small molecules) for engaging these targets, the development of injectable LAD technologies for controlled and sustained delivery of ocular therapeutics remains an underserved medical need.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates polymer nanoparticle (PNP) hydrogels formed through the interactions of poly(ethylene glycol)-block-poly(lactic acid) (PEG-PLA) nanoparticles (NPs) and dodecyl-modified hydroxypropyl methylcellulose polymers (HPMC-C₁₂).

FIG. 2 is a schematic illustration of a PNP hydrogel for treating ocular disease, prepared by mixing PEG-PLA NPs, HPMC-C₁₂, and an ocular therapeutic.

FIG. 3 is a schematic illustration of formation of a localized depot in the vitreous humor (VH) of an eye following intravitreal injection (ITV) of the PNP hydrogel.

FIG. 4 shows a schematic of the preparation of bimatoprost-loaded PNP hydrogels. When solutions of PEG-PLA nanoparticles, HPMC-C₁₂, and bimatoprost are mixed, cargo-loaded PNP hydrogels are formed.

FIG. 5 shows a schematic of the prolonged drug release from a PNP hydrogel placed in the VH. PNP hydrogels loaded with bimatoprost are shear-thinning and self-healing, enabling ITV injection and formation of a sustained delivery depot for extended release of bimatoprost in the VH.

FIGS. 6A-6D are a series of images showing how PNP hydrogels are prepared by first (i) loading PEG-PLA nanoparticles and cargo (blue dye) into one syringe (left) and HPMC-C₁₂ into another (right), (ii) fitting an elbow mixer and mixing back and forth to yield (iii) a homogeneous gel that is (iv) pre-loaded into a syringe for injection.

FIG. 7A is a graph showing the oscillatory shear rheology of PNP-2-10 hydrogels (i.e., 2 wt % HPMC-C₁₂+10 wt % PEG-PLA nanoparticles) with or without bimatoprost (0.25 mg mL⁻¹). The graph of FIG. 7A shows G′ (storage modulus) dominates over G″ (loss modulus), indicating robust solid-like properties that are not impacted by drug cargo.

FIG. 7B is a graph showing that low-to-high shear rheology reveals robust yield stress behavior.

FIG. 7C is a graph showing that high-to-low shear rheology reveals shear-thinning behavior.

FIG. 7D is a graph showing step-shear rheology measurements demonstrating that PNP hydrogel viscosity drops under high shear (10 s⁻¹) and recovers rapidly at low shear (1 s⁻¹) over repeated cycles.

FIG. 7E is a graph showing that analysis of the flow sweep behavior yields consistency index (K) and shear-thinning parameter (n) that indicate PNP hydrogels fall into the domain of injectability by healthcare professionals (K=195.5 and 85.62 Pa s⁻¹ and n=0.27 and 0.29 for hydrogels prepared with and without bimatoprost, respectively).

FIG. 8 is a schematic showing that the PNP hydrogel (50 μL) is easily injected through a 30-gauge needle into VH mimic and self-heals to form a solid depot.

FIG. 9A is a series of images taken at various timepoints showing that PNP hydrogels slow the release kinetics of fluorescein (0.25 mg mL⁻¹) in a VH mimic compared with a bolus of fluorescein prepared in PBS.

FIG. 9B is a graph showing the release of fluorescein from the PNP hydrogel and bolus. Release of fluorescein was quantified using ImageJ software and fit with a one-phase exponential decay in GraphPad Prism to determine a half-life of release from each vehicle (PNP hydrogel or PBS bolus).

FIG. 9C is a series of images taken at various timepoints showing that PNP hydrogels slow the release kinetics of albumin-FITC (10 mg mL⁻¹) in a VH mimic compared with a bolus of albumin-FITC prepared in PBS.

FIG. 9D is a graph showing the release of albumin-FITC from the PNP hydrogel and bolus. Release of albumin-FITC was quantified using ImageJ software and fit with a one-phase exponential decay in GraphPad Prism to determine a half-life of release from each vehicle (PNP hydrogel or PBS bolus).

FIG. 10A is a schematic of two static release assays wherein 100 μL of PNP hydrogel with bimatoprost cargo (0.25 mg mL⁻¹) was injected into the bottom of a capillary or dialysis tube and PBS buffer added. Buffer was sampled over time to quantify bimatoprost release.

FIG. 10B is a graph showing the quantification of bimatoprost release over time (n=3) as determined by LC-MS. Data are shown as mean±SD and fit with a one-phase decay in GraphPad Prism and half-lives calculated.

FIG. 10C is a schematic of a dynamic release assay with 400-DS apparatus 7 wherein 100 μL of PNP hydrogel with bimatoprost (0.74 mg mL⁻¹) was injected into a mesh basket and PBS buffer added. The basket was dipped at 1 cycle per minute and buffer sampled over time.

FIG. 10D is a graph showing the quantification of bimatoprost release over time (n=2) as determined by RP-CAD. Data are shown as individual points and fit with a one-phase exponential decay in GraphPad Prism and half-life calculated.

FIG. 11A is a timeline of rabbit tolerability and PK study. PNP hydrogel alone (Gel) or with bimatoprost (0.16 mg mL⁻¹) (Gel+Bim) was injected intravitreally into both eyes of rabbits on day 1 (50 μL injection). Ocular tolerability was evaluated over time by ophthalmic exam (OE) with tonometry. Wide-field color fundus and ultrasound imaging were used to monitor the appearance of the hydrogel and SD-OCT was used to qualitatively assess retinal layer thickness. Globes and VH were collected at terminal time points on days 29 and 57 for histology and PK analysis.

FIG. 11B is a series of representative color fundus images that show clear depot formation on days 3 and 15 with substantial degradation of the depot on day 57 (O.D.=oculus dexter, O.S.=oculus sinister).

FIG. 11C is a graph showing bimatoprost levels in VH. Bimatoprost concentration was quantified by LC-MS at terminal time points on days 29 (n=3) and 57 (n=4).

FIG. 11D is a graph showing the VH half-life of bimatoprost dosed using PNP hydrogel. The half-life was calculated using an administered dose of 8 μg bimatoprost in an estimated 1.5 mL VH and one-phase decay were fit in GraphPad Prism. VH half-life of bimatoprost alone was predicted using a pharmacokinetic model based on its physiochemical parameters.

FIG. 12A is a graph showing that intraocular pressure (IOP) measurements for eyes injected with PNP hydrogel alone (Gel), or with bimatoprost (0.16 mg mL⁻¹) (Gel+Bim), fell within the average range (10-20 mmHg). IOP elevation following administration, and decrease on days 3 and 8, were expected procedure-related trends following standard ITV injection. Data shown as mean±SD.

FIG. 12B is a chart showing that the means of clinical scores for several OE parameters showed presence of cells and cell-like material in the VH that mostly resolved and trended towards recovery at the end of the study (AC=aqueous chamber, V=vitreous).

FIG. 12C shows a series of representative SD-OCT images that demonstrate subjectively no changes in retinal thickness over time but identify an accumulation of hyperreflective debris within the posterior VH (blue arrows) at the terminal timepoint, presumably a product of PNP hydrogel breakdown (O.D.=oculus dexter).

FIG. 13A shows a representative image of H&E staining of the full rabbit eye (scale bar=2500 μm) with an affected region extending from the ciliary body and along the ventral peripheral retina. Minimal to mild foreign body response in the VH and along the ventral retina was observed microscopically in response to PNP hydrogel.

FIGS. 13B and 13C show increasingly higher magnifications of an affected region of the image of the H&E-stained rabbit eye shown in FIG. 13A. Foamy macrophages can be observed.

### DETAILED DESCRIPTION

Injectable hydrogels are promising candidates for ocular LAD systems, as these technologies possess numerous unique and desirable features. The high water content and tunable mechanical properties of hydrogels afford exceptional modularity and biocompatibility. Hydrogel systems that employ mild gelation mechanisms, such as supramolecular interactions or ionic, pH, and temperature-triggered interactions, maintain an aqueous environment and promote payload stability, contributing to the versatility of these materials in biologic applications. Although numerous injectable hydrogel formulations are currently in development as long-acting depots in the eye, none have been approved to date. Barriers to success include a high degree of burst release, poorly matched timescales of drug release and depot degradation (potentially resulting in buildup of depot components), complex manufacturing, and a lack of broad compatibility with various payloads. In addition, manufacturers are often required to demonstrate safety over an extended period of time since the prolonged presence, or potential accumulation of polymer matrix with repeated dosing, may elicit vitreous haze, foreign body responses, retinal toxicities, and an increased risk of visual disturbance.

To address these and other challenges, the present technology provides compositions and methods for delivery of ocular therapeutics, such as prostaglandin analogs (PGAs) and others. In some embodiments, for example, the disclosure provides a composition for treating an ocular disease or condition (e.g., glaucoma), where the composition includes a dynamic hydrogel composed of a polymer (e.g., a hydrophobically modified cellulose derivative) and a plurality of nanoparticles (e.g., amphiphilic polymeric nanoparticles). The polymer can be non-covalently crosslinked with the plurality of nanoparticles, thus conferring shear-thinning, self-healing, and/or viscoelastic properties to the dynamic hydrogel. The composition can further include an ocular therapeutic encapsulated by the dynamic hydrogel, such as a PGA. In some embodiments, the ocular therapeutic is encapsulated via hydrophobic interactions between the ocular therapeutic and hydrophobic surfaces of the nanoparticles. The ocular therapeutic can be gradually released from the dynamic hydrogel via erosion of the dynamic hydrogel in vivo. Accordingly, upon administration of the composition to the subject, the composition can provide sustained, controlled release of the ocular therapeutic over a desired treatment period at a rate that is effective for treating the disease or condition of the eye. For example, the compositions herein can be designed to provide continuous delivery of an ocular therapeutic for upwards of two months from a single administration.

The embodiments of the present technology can provide numerous advantages compared to conventional therapeutic products and treatment approaches. For instance, conventional hydrogel-based depot technologies typically exhibit several critical shortcomings, including complicated manufacturing, poor formulation stability, challenging administration, burst release that can contribute to poor tolerability of the therapy, and insufficiently slow release to enable appropriately long-acting therapies. In contrast to conventional covalently crosslinked hydrogels, the dynamic hydrogels of the present technology are formed through strong yet dynamic physical interactions. As a result, these materials can address the shortcomings of other hydrogel-based depot technologies by exhibiting: (i) mild formulation requirements favorable for facile formulation with therapeutic cargo, such as an ocular therapeutic, and for maintaining drug stability during manufacturing and storage; (ii) shear-thinning properties allowing for straightforward injectability through standard syringes and needles (including ITV needles) thus improving patient convenience; (iii) rapid self-healing of hydrogel structure and depot formation to avoid burst release of the therapeutic cargo, thus providing excellent tolerability by maintaining consistent slow release to circumvent undesirable side effects; (iv) sufficiently high yield stress to form a robust depot that persists under the normal stresses of the VH space following administration; (v) prolonged delivery of therapeutic cargo allowing for continuous delivery over clinically desirable timeframes; (vi) biodegradability; (vii) non-immunogenicity, as well as not promoting immune responses to the encapsulated cargo, and/or (viii) similar properties to that of the VH (e.g., density, stiffness, etc.), thus providing good compatibility for implantation in the VH.

Embodiments of the present disclosure will be described more fully hereinafter with reference to the accompanying drawings in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology. Embodiments under any one heading may be used in conjunction with embodiments under any other heading.

### I. Dynamic Hydrogels

The present technology utilizes dynamic hydrogels that can serve as a versatile platform for controlled release of therapeutic cargo, such as the ocular therapeutics described in Section II below. In some embodiments, the dynamic hydrogels exhibit dynamic behavior, such as shear-thinning behavior, self-healing behavior, and/or highly tunable viscoelastic mechanical properties. The shear-thinning, self-healing, and/or viscoelastic properties of the dynamic hydrogels can result from non-covalent, supramolecular interactions between the components of the hydrogel (e.g., polymers and nanoparticles, as described further below). The non-covalent interactions can include physical crosslinking, which may encompass various types of crosslinking arising from weak physical interactions such as hydrogen bonding, hydrophobic interactions, ionic interactions, van der Waals interactions, host-guest interactions, crystal formation, physical entanglement, or combinations thereof. The non-covalent interactions can allow for the formation of dynamic, reversible crosslinks between components of the hydrogel that are capable of dissociating and reforming, e.g., spontaneously and/or in response to applied stress.

The dynamic hydrogels described herein can provide many advantages for therapeutic applications. For instance, the dynamic hydrogels described herein can exhibit high drug loading capacity, gentle conditions for encapsulation of biologic cargo, sustained delivery of cargo, and/or mechanical tunability. However, unlike traditional covalently crosslinked hydrogels, the dynamic hydrogels herein can be easily administered via techniques such as direct injection, catheter delivery, spreading, or spraying, due to their shear-thinning and/or self-healing properties. Additionally, the dynamic hydrogels herein can exhibit unique dynamic network rearrangements that provide highly tunable release characteristics for the therapeutic cargo. The dynamic hydrogels provided herein can also be synthesized in a straight-forward, cost-effective manner that is easily scalable.

### A. Polymer Nanoparticle Hydrogels

In some embodiments, the dynamic hydrogels described herein are polymer nanoparticle (PNP) hydrogels. PNP hydrogels are a type of supramolecular hydrogel formed from non-covalent interactions between polymers and nanoparticles. A PNP hydrogel can self-assemble rapidly upon mixing of a polymer solution with a nanoparticle solution. Self-assembly of the PNP hydrogel network can occur when polymers are linked together by adsorption of segments of the polymer chains onto the surfaces of the nanoparticles through multivalent, transient interactions. PNP hydrogel formation can be an entropy-driven process in which solvent molecules (e.g., water) solvating the polymer chains and nanoparticle surfaces are released into the bulk solution upon binding of the polymer chains to the nanoparticle surfaces, thus producing large gains in translational entropy. The interactions between the polymers and nanoparticle surfaces can be transient and reversible, thus allowing the PNP hydrogel to flow under applied shear stress, followed by rapid self-healing when the stress is relaxed.

The PNP hydrogels described herein can be composed of any suitable combination of polymers and nanoparticles that are capable of interacting non-covalently with each other to form crosslinks with the desired dynamic behavior. In some embodiments, the nanoparticle and polymer are selected to have a sufficiently strong affinity to produce efficient crosslinking. That is, the free energy gain (c) resulting from the adsorption of a polymer chain to the surface of a nanoparticle can be greater than or comparable to the thermal energy (k_{B}T). In addition, the average number of interactions per polymer chain and particle can be greater than 2 to achieve percolation of the hydrogel network. Moreover, to favor polymer bridging of multiple nanoparticles (as opposed to polymer wrapping around individual particles), the nanoparticle diameter can be comparable to or less than the persistence length of the polymer chains. When some or all of these criteria are met, the nanoparticles can serve as crosslinkers between the polymer chains, while the polymer chains can bridge many different particles, thus enabling hydrogel formation. In some embodiments, the modulus (G) of the PNP hydrogel is related to the number of dynamic hydrogel interactions per unit volume (n) and the energy associated with each interaction (αk_{B}T) according to the following relation: G≈nαk_{B}T. In some embodiments, the nanoparticle surfaces are hydrophobic, such that the adsorption of the polymer chains to the nanoparticle surfaces are at least partially influenced by the general level of hydrophobicity along the polymer chain (e.g., the size and/or number of hydrophobic groups attached to the polymer chain).

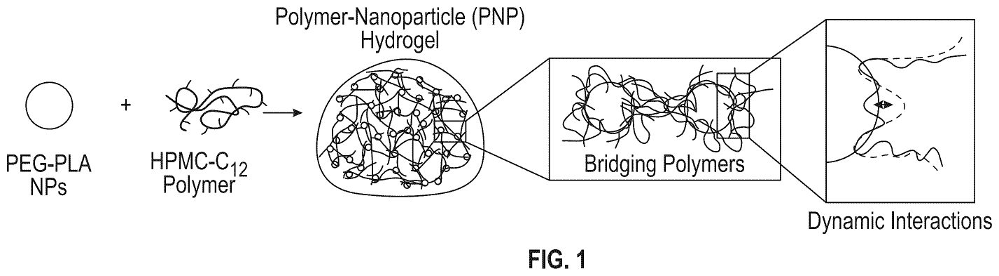

For example, as shown in FIG. 1, the PNP hydrogels described herein can be formed through dynamic interactions between hydrophobically-modified cellulose derivatives and nanoparticles, such as dodecyl-modified hydroxypropylmethylcellulose (HPMC-C₁₂) and biodegradable polymeric nanoparticles composed of poly(ethylene glycol)-block-poly(lactic acid) (PEG-PLA). The polymers can bridge between nanoparticles and dynamically interact with the nanoparticle surfaces. Additional examples of nanoparticles and polymers suitable for use in the PNP hydrogels herein are provided in Sections I.A.1 and I.A.2 below, respectively.

The PNP hydrogels described herein can be differentiated from conventional drug delivery systems that include nanoparticles embedded in a covalently crosslinked hydrogel. Such conventional systems typically include gel-forming polymers that are covalently crosslinked with each other to form the gel network, while the nanoparticles serve as an optional additive that plays no role in gel formation, and thus can be freely substituted with other additives or omitted altogether. In contrast, the PNP hydrogels herein may be specifically formed through the interactions between the nanoparticles and polymers. In some embodiments, the polymers and nanoparticles used in the PNP hydrogels herein each independently do not form a gel alone, or are not used at concentrations where the polymer alone or the nanoparticle alone form a gel, such that gel formation occurs only when the polymer and nanoparticle are combined.

In some embodiments, the PNP hydrogels herein include one or more polymers combined with one or more nanoparticles, such that the loss modulus of a solution of the one or more polymers and the loss modulus of a solution of the one or more particles are each greater than their respective storage moduli at an angular frequency within a range from 0.1 rad/s to 100 rad/s (e.g., 10 rad/s) as measured by oscillatory shear rheometry in the linear viscoelastic region. The storage modulus of the PNP hydrogel produced by combining the one or more polymers with the one or more particles may be greater than the loss modulus of the PNP hydrogel at an angular frequency within a range from 0.1 rad/s to 100 rad/s (e.g., 10 rad/s) as measured by oscillatory shear rheometry in the linear viscoelastic region. In some embodiments, the dynamic shear viscosity of the PNP hydrogel at a shear rate within a range from 0.1 s⁻¹ to 100 s⁻¹ (e.g., 10 s⁻¹) is greater than the sum of the dynamic shear viscosity of the solution of the one or more polymers and dynamic shear viscosity of the solution of the one or more nanoparticles at the shear rate within the range from 0.1 s⁻¹ to 100 s⁻¹. For example, the dynamic shear viscosity of the PNP hydrogel can be greater than the sum of the dynamic shear viscosities of the polymer solution and the nanoparticle solution by a multiplicative factor within a range from 2 to 100,000, 2 to 1000, 2 to 100, or 2 to 10.

The PNP hydrogels described herein can include any concentration of polymers and nanoparticles suitable for providing desired hydrogel properties. For instance, higher polymer concentrations can produce PNP hydrogels with a higher stiffness and/or slower degradation rate. Higher nanoparticle concentrations can produce PNP hydrogels with a higher viscosity, stiffness, and yield stress, and/or slower degradation rate. The hydrogel properties may depend not only on the overall amount of solid content in the hydrogel, but also on the stoichiometry of polymer content to nanoparticle content. For example, increasing the nanoparticle concentration at a constant polymer concentration can produce a hydrogel having a more solid-like rheological response (e.g., lower tan delta), increased strain-to-yield, and increased yield stress. Increasing the polymer concentration at a constant nanoparticle concentration can produce a hydrogel having a more liquid-like rheological response (e.g., higher tan delta and greater frequency dependency of the storage modulus) and reduced strain-to-yield.

In some embodiments, the PNP hydrogels described herein include at least 0.25 wt %, 0.5 wt %, 0.75 wt %, 1 wt %, 1.5 wt %, 2 wt %, 2.5 wt %, 3 wt %, 3.5 wt %, 4 wt %, 4.5 wt %, or 5 wt % polymer; and/or at least 0.5 wt %, 1 wt %, 2 wt %, 3 wt %, 4 wt %, 5 wt %, 6 wt %, 7 wt %, 8 wt %, 9 wt %, 10 wt %, 12 wt %, or 15 wt % nanoparticles. Alternatively or in combination, the concentration of polymer within the PNP hydrogel can be within a range from 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %; and/or the concentration of nanoparticles within the PNP hydrogel can be within a range from 1 wt % to 12 wt %, 1 wt % to 10 wt %, 1 wt % to 8 wt %, 1 wt % to 5 wt %, 1 wt % to 3 wt %, 3 wt % to 12 wt %, 3 wt % to 10 wt %, 3 wt % to 8 wt %, 3 wt % to 5 wt %, 5 wt % to 12 wt %, 5 wt % to 10 wt %, 5 wt % to 8 wt %, 8 wt % to 12 wt %, 8 wt % to 10 wt %, or 10 wt % to 12 wt %. The nomenclature “X-Y hydrogel” or “X:Y hydrogel” is used herein to refer to a hydrogel having X wt % polymer and Y wt % nanoparticles.

In some embodiments, the PNP hydrogels herein are prepared by simple mixing of the polymers, nanoparticles, therapeutic cargo, and any optional additives. For example, the PNP hydrogel can be prepared by forming a polymer solution (e.g., by dissolving the polymer in an aqueous solvent such as water or a buffered solution such as phosphate-buffered saline (PBS)), forming a nanoparticle solution (e.g., by suspending the nanoparticles in an aqueous solvent), and forming a solution containing the therapeutic cargo (e.g., by dissolving or suspending the therapeutic cargo in an aqueous solvent). The solutions can then be combined, optionally with external agitation, to form the PNP hydrogel including the therapeutic cargo.

### 1. Nanoparticles

The PNP hydrogels described herein can include a plurality of nanoparticles. The nanoparticles can be any suitable shape, such as spheres, cubes, rods, tubes, plates, fibers, etc. The nanoparticles can have a mean particle size (e.g., diameter) within a range from 1 nm to 1000 nm, 1 nm to 500 nm, 1 nm to 250 nm, 1 nm to 150 nm, 1 nm to 100 nm, 1 nm to 50 nm, 1 nm to 25 nm, 1 nm to 10 nm, 10 nm to 1000 nm, 10 nm to 500 nm, 10 nm to 250 nm, 10 nm to 150 nm, 10 nm to 100 nm, 10 nm to 50 nm, 10 nm to 25 nm, 25 nm to 1000 nm, 25 nm to 500 nm, 25 nm to 250 nm, 25 nm to 150 nm, 25 nm to 100 nm, 25 nm to 50 nm, 50 nm to 1000 nm, 50 nm to 500 nm, 50 nm to 250 nm, 50 nm to 150 nm, 50 nm to 100 nm, 100 nm to 1000 nm, 100 nm to 500 nm, 100 nm to 250 nm, 100 nm to 150 nm, 150 nm to 1000 nm, 150 nm to 500 nm, 150 nm to 250 nm, 250 nm to 1000 nm, 250 m to 500 nm, or 500 nm to 1000 nm. In some embodiments, the nanoparticles have a mean particle size less than or equal to 500 nm, 250 nm, 150 nm, 140 nm, 130 nm, 120 nm, 110 nm, 100 nm, 90 nm, 80 nm, 70 nm, 60 nm, 50 nm, 40 nm, 30 nm, 20 nm, or 10 nm. As described herein, to facilitate hydrogel formation, the mean particle size of the nanoparticles can be similar to or less than the persistence length of the polymer in the PNP hydrogel, such as less than or equal to 125%, 110%, 100%, 90%, 80%, 70%, 60%, 50%, 40%, 30%, 20%, or 10% of the persistence length of the polymer. As used herein, “mean particle size” may refer to the statistical mean particle size (e.g., diameter) of the particles in the PNP hydrogel composition. The diameter of an essentially spherical particle may refer to the physical or hydrodynamic diameter. The diameter of a non-spherical particle may refer to the hydrodynamic diameter or to the largest linear distance between two points on the surface of the particle. Mean particle size can be measured using methods known in the art, such as dynamic light scattering.

The nanoparticles can be made out of a single material or can be made out of a combination of multiple different materials (e.g., two, three, four, five, or more different materials). The material(s) can be biodegradable and/or biocompatible. For example, in some embodiments, the nanoparticles are made partially or entirely out of one or more biodegradable and/or biocompatible polymers. Generally, biodegradable polymers can degrade by enzymatic hydrolysis, exposure to water in vivo, surface erosion, and/or bulk erosion. Biodegradable polymers can include synthetic polymers, naturally occurring polymers, or combinations thereof. Examples of synthetic biodegradable polymers include polyhydroxy acids (e.g., poly(lactic acid), poly(glycolic acid)), polyanhydrides, poly(ortho) esters, polyesters, polyurethanes, poly(butyric acid), poly(valeric acid), poly(caprolactone), poly(hydroxybutyrate), poly(lactide-co-glycolide), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), and combinations (e.g., mixtures, copolymers) thereof. Examples of naturally occurring biodegradable polymers include polysaccharides (e.g., cellulose, alginate, collagen, chitosan, hyaluronic acid, starch, agarose, agar, xanthan gum), proteins (e.g., collagen, fibrin, albumin, zein, gelatin), derivatives thereof (e.g., derivatives of cellulose such as cellulose nanocrystals, cellulose nanofibers), and combinations thereof.

Alternatively or in combination, the nanoparticles can be made partially or entirely out of one or more non-biodegradable polymers. Examples of non-biodegradable polymers include polystyrenes, polyalkylene glycols, poly(meth)acrylates, poly(meth)acrylamides, polyalkylenes (e.g., polyethylene, polyvinyls, poly(vinyl acetate), poly(ethylene terephthalate)), and combinations thereof.

The polymer(s) used to form the nanoparticles herein can have any suitable molecular weight, such as a molecular weight (e.g., number-average molecular weight (Mn)) within a range from 500 Da to 10,000 kDa, 1 kDa to 1000 kDa, or 10 kDa to 100 kDa. As used herein, “molecular weight” may refer to the relative average chain length of the bulk polymer, and can be estimated or characterized in various ways including gel permeation chromatography (GPC) and capillary viscometry. GPC molecular weights are reported as the number-average molecular weight (Mn) as opposed to the weight-average molecular weight (Mw). Capillary viscometry provides estimates of molecular weight (Me) as the inherent viscosity determined from a dilute polymer solution using a particular set of concentration, temperature, and solvent conditions.

In some embodiments, the nanoparticles are made partially or entirely out of one or more inorganic materials, such as clays (e.g., silicates) or other types of minerals (e.g., sulfides, oxides, halides, carbonates, sulfates, phosphates, apatites), or combinations thereof. Alternatively or in combination, the nanoparticles can be made partially or entirely out of one or more metals, such as gold, silver, copper, platinum, palladium, ruthenium, or combinations thereof. Optionally, the nanoparticles can be made partially or entirely out of carbon nanotubes (e.g., single-walled or multi-walled nanotubes), graphene, graphene oxide, or other ultrathin single crystals, including black phosphorous and boron based nanosheets.

In some embodiments, the nanoparticles are core-shell particles (also known as “core-corona particles”). A core-shell particle can have a core containing or formed from a first material, and a shell or corona containing or formed from a second, different material. For example, a core-shell particle can include at least two polymers, such that the core is made from a first polymer, and the shell or corona is made from a second, different polymer. As another example, the core-shell particle can include a single block copolymer, such that the core is made from a first block of the block copolymer, and the shell or corona can be made from a second block of the block copolymer. In some embodiments, one or both of the components of the core-shell particle is a non-polymeric material.

A core-shell particle can be composed of two compositionally disparate phases, of which one (either the core or shell/corona) is hydrophobic and the other (core or shell/corona) is hydrophilic. Suitable hydrophobic components include polyamides (e.g., poly(amino acids)), polyesters (e.g., poly(lactic acid), poly(caprolactone)), polypropylene oxides, polystyrenes, and combinations thereof. Suitable hydrophilic components include polysaccharides, proteins, polyamides (e.g., poly(amino acids)), naturally occurring polymers, synthetic polymers, and combinations thereof. Suitable block copolymers include combinations of polyethylene glycol and polyesters (e.g., PEG-PLA, poly(ethylene glycol)-block-poly(caprolactone) (PEG-PCL)) and combinations of polyethylene glycol and polypropylene glycol (e.g., poloxamers). In some embodiments, the core-shell particle is composed of an amphiphilic polymer including (1) one or more hydrophobic polymers selected from polyanhydrides, poly(ortho) esters, polyesters, polyurethanes, and/or copolymers thereof, and (2) one or more hydrophilic polymers selected from polysaccharides, proteins, poly(amino acids), and/or polyalkylene oxides.

Alternatively, the nanoparticles can be homogenous nanoparticles. A homogenous nanoparticle can be uniformly formed from a single material, or can be formed from multiple materials that are not separated into disparate phases within the particle as in core-shell particles.

The nanoparticles can be prepared using techniques known in the art. The technique to be used can depend on a variety of factors, including the materials used to form the nanoparticles, the desired size range of the resulting nanoparticles, and suitability for the material to be encapsulated. Examples of suitable techniques include, but are not limited to, solvent evaporation, solvent removal, hot melt microencapsulation, spray drying, phase inversion, polyelectrolyte condensation, single and double emulsion (e.g., probe sonication), nanoparticle molding, and electrostatic self-assembly.

The concentration of the nanoparticles in the PNP hydrogel can be varied to produce the desired hydrogel properties. In some embodiments, for example, the concentration of the nanoparticles in the PNP hydrogel is within a range from 1 wt % to 15 wt %, 2 wt % to 12 wt %, 3 wt % to 10 wt %, 5 wt % to 8 wt %, 5 wt % to 15 wt %, 5 wt % to 10 wt %, 10 wt % to 15 wt %, or 10 wt % to 12 wt %. The concentration of the nanoparticles in the PNP hydrogel can be about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, or about 15 wt %. In some embodiments, the concentration of the nanoparticles in the PNP hydrogel can be greater than or equal to 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, or 14 wt %. Alternatively or in combination, the concentration of the nanoparticles in the PNP hydrogel can be less than or equal to 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, or 15 wt %.

### 2. Polymers

The PNP hydrogel can be formed when the nanoparticles are mixed with and interact with one or more polymers. The shear-thinning and/or self-healing properties of the PNP hydrogel can be derived from reversible, non-covalent interactions between the nanoparticles and the polymer chains, as described herein. The PNP hydrogel can include a single type of polymer or can include a combination of multiple different polymers (e.g., two, three, four, five, or more different polymers). The polymer(s) can be biodegradable and/or biocompatible. The polymer(s) can include naturally occurring polymers, synthetic polymers, or derivatives or combinations thereof. Examples of naturally occurring polymers include polysaccharides (e.g., cellulose, alginate, collagen, chitosan, hyaluronic acid, starch, agarose, agar, xanthan gum), proteins (e.g., collagen, fibrin, albumin, zein, gelatin), and combinations thereof. Examples of synthetic polymers include polyacrylamide, poly(lactic acid), polyethylene glycol, polyethylene glycol-co-propylene glycol (PEO-PPO), poly(acrylates) (e.g., poly(2-hydroxyethyl methacrylate)), and combinations thereof. In some embodiments, the PNP hydrogel includes a derivative of a naturally occurring polymer, such as a cellulose derivative. Examples of cellulose derivatives include hydroxypropylmethylcellulose (HPMC), hydroxyethyl cellulose (HEC), hydroxypropylcellulose (HPC), ethylcellulose (EC), methylcellulose (MC), hydroxyethylmethylcellulose (HEMC), carboxymethylcellulose (CMC), carboxymethyl ethyl cellulose (CMEC), and combinations thereof.

In some embodiments, the PNP hydrogels herein include at least one polymer that is modified with a hydrophobic moiety. Hydrophobic modification of polymers may increase the energy associated with each polymer nanoparticle interaction (αk_{B}T), thereby increasing the modulus of the dynamic hydrogel given the same number of interactions per unit volume. Such modification may facilitate favorable interactions between the hydrophobic moiety on the polymer chain and the hydrophobic core of the nanoparticle, thereby enhancing the adsorption energy of the polymer to the nanoparticles. The hydrophobic moiety can include a plurality of carbon atoms (e.g., from 2 to 50 carbon atoms, 2 to 30 carbon atoms, or 2 to 18 carbon atoms), and can be a saturated molecule or an unsaturated molecule. Examples of hydrophobic moieties that may be used include, but are not limited to, alkyl moieties (e.g., C4 to C18 alkyls, such as butyl (—C4), hexyl (—C6), octyl (—C8), decyl (—C10), dodecyl (—C₁₂), tetradecyl (—C14), pentadecyl (—C15), hexadecyl (—C16), heptadecyl (—C17), octadecyl (—C18)), alkenyl moieties (e.g., oleyl, linoleyl), aryl moieties (e.g., phenyl, benzyl, pyryl, naphthyl, anthracene), and cycloalkyl moieties (e.g., adamantyl, cyclohexyl, cholesterol). In some embodiments, the degree of modification of the polymer (e.g., percentage of reactive groups on the polymer have been functionalized with the hydrophobic moiety) is within a range from 1% to 50%, 5% to 30%, 5% to 25%, or 10% to 15%. For example, the degree of modification can be about 5%, 10%, 15%, 20%, or 25%.

The concentration of the polymer(s) in the PNP hydrogel can be varied to produce the desired hydrogel properties (e.g., stiffness, storage modulus, degradation rate). In some embodiments, for example, the concentration of the polymer(s) in the PNP hydrogel is within a range from 0.25 wt % to 10 wt %, 0.5 wt % to 5 wt %, 0.5 wt % to 2 wt %, 1 wt % to 5 wt %, or 1 wt % to 2 wt %. The concentration of the polymer(s) in the PNP hydrogel can be about 0.1 wt %, 0.25 wt %, 0.5 wt % 0.75 wt %, 1 wt %, 1.25 wt %, 1.5 wt %, 1.75 wt %, 2 wt %, 2.25 wt %, 2.5 wt %, 2.75 wt %, 3 wt %, 3.5 wt %, 4 wt %, 4.5 wt %, or 5 wt %. In some embodiments, the concentration of the polymer(s) in the PNP hydrogel can be greater than or equal to 0.25 wt %, 0.5 wt % 0.75 wt %, 1 wt %, 1.25 wt %, 1.5 wt %, 1.75 wt %, 2 wt %, 2.25 wt %, 2.5 wt %, 2.75 wt %, 3 wt %, 3.5 wt %, 4 wt %, or 4.5 wt %. Alternatively or in combination, the concentration of the polymer(s) in the PNP hydrogel can be less than or equal to 0.5 wt % 0.75 wt %, 1 wt %, 1.25 wt %, 1.5 wt %, 1.75 wt %, 2 wt %, 2.25 wt %, 2.5 wt %, 2.75 wt %, 3 wt %, 3.5 wt %, 4 wt %, 4.5 wt %, or 5 wt %.

### 3. Additional Components

The PNP hydrogels herein can optionally include one or more additional components to facilitate gel formation and/or modify the properties of the hydrogel. For example, the PNP hydrogels herein can include at least one enhancer compound that enhances the interactions between the polymers and nanoparticles, e.g., by providing bridging-type non-covalent interactions between the polymers and nanoparticles. In some embodiments, a portion of an enhancer compound interacts non-covalently with the polymer and a second portion of the enhancer compound interacts non-covalently with the nanoparticle. Non-limiting examples of such interactions include ionic interactions such as cationic/anionic interactions, electrostatic interactions, and hydrogen bonding interactions.

For example, in embodiments where the polymer is negatively charged at physiological pH (e.g., hyaluronic acid, carboxymethyl cellulose), a cationic surfactant can be used to enhance adsorption of the anionic polymer to the nanoparticles via electrostatic interactions. Examples of positively charged surfactants include cetyltrimethylammonium bromide (CTAB), cetyltrimethylammonium iodide, cetyltrimethylammonium fluoride, and cetyltrimethylammonium chloride. Conversely, in embodiments where the polymer is positively charged at physiological pH (e.g., chitosan, aminopolysaccharides, poly(lysine), cationic acrylate polymers, cationic vinyl polymers), an anionic surfactant can be used to enhance adsorption of the cationic polymer to the nanoparticles via electrostatic interactions. Examples of negatively charged surfactants include sodium dodecyl sulfate, sodium stearate, and charged fatty acid surfactants.

In some embodiments, molecular recognition between at least two compounds can provide the enhancement. For example, the adsorption of polymers, such as polysaccharides, to nanoparticles can be enhanced by an enhancer compound which includes a carbohydrate in one portion of the enhancer and a polymer tail that interacts with the nanoparticle.

The concentration of the enhancer compound can be varied to produce the desired effect on hydrogel formation. In some embodiments, for example, the concentration of the enhancer compound in the PNP hydrogel is within a range from 0.25 wt % to 10 wt %, 0.5 wt % to 5 wt %, 0.5 wt % to 2 wt %, 1 wt % to 5 wt %, or 1 wt % to 2 wt %. The concentration of the enhancer compound in the PNP hydrogel can be about 0.1 wt %, 0.25 wt %, 0.5 wt % 0.75 wt %, 1 wt %, 1.25 wt %, 1.5 wt %, 1.75 wt %, 2 wt %, 2.25 wt %, 2.5 wt %, 2.75 wt %, 3 wt %, 3.5 wt %, 4 wt %, 4.5 wt %, or 5 wt %. In some embodiments, the concentration of the enhancer compound in the PNP hydrogel can be greater than or equal to 0.25 wt %, 0.5 wt % 0.75 wt %, 1 wt %, 1.25 wt %, 1.5 wt %, 1.75 wt %, 2 wt %, 2.25 wt %, 2.5 wt %, 2.75 wt %, 3 wt %, 3.5 wt %, 4 wt %, or 4.5 wt %. Alternatively or in combination, the concentration of the enhancer compound in the PNP hydrogel can be less than or equal to 0.5 wt % 0.75 wt %, 1 wt %, 1.25 wt %, 1.5 wt %, 1.75 wt %, 2 wt %, 2.25 wt %, 2.5 wt %, 2.75 wt %, 3 wt %, 3.5 wt %, 4 wt %, 4.5 wt %, or 5 wt %. Optionally, the PNP hydrogel may not include any enhancer compounds.

### B. Hydrogel Properties

The dynamic hydrogels described herein (e.g., the PNP hydrogels of Section I.A) can exhibit favorable physical and biological properties that contribute to their efficacy as drug delivery platforms. The properties of the dynamic hydrogels herein can be tuned in various ways, such as by modifying the types of components used to form the hydrogel (e.g., polymers, nanoparticles, and/or additional components as previously described in Section I.A; and/or the therapeutic cargo carried by the hydrogel as described below in Section II), the concentrations of the components, and/or the chemical functionalities of the components. Accordingly, the properties of the dynamic hydrogels herein can be adapted to the particular therapeutic application, such as forming a stable and/or persistent depot when delivered in vivo, providing a desired release profile for the therapeutic cargo (e.g., short-term release versus long-term release), providing a desired release mechanism for the therapeutic cargo (e.g., diffusion-based release versus erosion-based release), compatibility with a desired route of administration (e.g., injecting, infusing, spraying, spreading), biodegradability, biocompatibility, and/or allowing for cellular infiltration. Any reference herein to a property of a dynamic hydrogel may refer to the property of the dynamic hydrogel without any therapeutic cargo (e.g., a PNP hydrogel composed only of polymers and nanoparticles), the property of the dynamic hydrogel including the therapeutic cargo (e.g., a PNP hydrogel including polymers, nanoparticles, and the encapsulated therapeutic cargo), or both, unless otherwise stated or otherwise evident from the context.

The storage modulus (G′) of the dynamic hydrogel can correlate to the overall stiffness of the hydrogel, which in turn can dictate the time scale of degradation of the hydrogel (e.g., hydrogels having a higher storage modulus may be stiffer and degrade more slowly than gels having a lower storage modulus). Accordingly, in embodiments where the therapeutic cargo of the dynamic hydrogel is released primarily or entirely via an erosion-based mechanism, the release rate of the therapeutic cargo can be tuned by adjusting the storage modulus of the hydrogel (e.g., a higher storage modulus can produce a slower degradation rate and thus a slower release rate of the therapeutic cargo, while a lower storage modulus can produce a higher degradation rate and thus a faster release rate of the therapeutic cargo). For example, in embodiments where the dynamic hydrogel is a PNP hydrogel, the storage modulus of the PNP hydrogel can be increased or decreased by increasing or decreasing the polymer concentration, and/or by increasing or decreasing the nanoparticle concentration. In some embodiments, the dynamic hydrogels herein have a storage modulus within a range from 1 Pa to 10,000 Pa, 1 Pa to 5000 Pa, 1 Pa to 2500 Pa, 1 Pa to 1000 Pa, 1 Pa to 500 Pa, 1 Pa to 200 Pa, 1 Pa to 10 Pa, 10 Pa to 10,000 Pa, 10 Pa to 5000 Pa, 10 Pa to 2500 Pa, 10 Pa to 1000 Pa, 10 Pa to 500 Pa, 10 Pa to 200 Pa, 10 Pa to 100 Pa, 10 Pa to 50 Pa, 50 P to 10,000 Pa, 50 Pa to 5000 Pa, 50 Pa to 2500 Pa, 50 Pa to 1000 Pa, 50 Pa to 500 Pa, 50 Pa to 200 Pa, 50 Pa to 100 Pa, 100 Pa to 10,000 Pa, 100 Pa to 5000 Pa, 100 Pa to 2500 Pa, 100 Pa to 1000 Pa, 100 Pa to 500 Pa, 100 Pa to 200 Pa, 200 Pa to 10,000 Pa, 200 Pa to 5000 Pa, 200 Pa to 2500 Pa, 200 Pa to 1000 Pa, 200 Pa to 500 Pa, 500 Pa to 10,000 Pa, 500 Pa to 5000 Pa, 500 Pa to 2500 Pa, 500 Pa to 1000 Pa, 1000 Pa to 10,000 Pa, 1000 Pa to 5000 Pa, 1000 Pa to 2500 Pa, 2500 Pa to 10,000 Pa, 2500 Pa to 5000 Pa, or 5000 Pa to 10,000 Pa. The storage modulus can be measured, for example, using an oscillatory shear test in a parallel plate rheometer at an angular frequency of 10 rad/s, a strain within the linear viscoelastic region of the hydrogel (e.g., 1% strain), and a temperature of 25° C.

The yield stress (Ty) of the dynamic hydrogel can correlate to the ability of the hydrogel to form and maintain a cohesive depot in vivo (e.g., materials lacking a yield stress may flow rather than forming a cohesive depot). The dynamic hydrogels herein can exhibit little or no flow when subjected to stresses below the yield stress. When subjected to stresses above the yield stress, the dynamic hydrogels can flow, corresponding to a significant drop in observed viscosity (e.g., a decrease of at least one or two orders of magnitude). In embodiments where the dynamic hydrogel is a PNP hydrogel, the yield stress can be increased or decreased by increasing or decreasing the nanoparticle concentration, respectively. In some embodiments, the dynamic hydrogels herein have a yield stress within a range from 0.1 Pa to 1000 Pa, 0.1 Pa to 500 Pa, 0.1 Pa to 200 Pa, 0.1 Pa to 100 Pa, 0.1 Pa to 50 Pa, 0.1 Pa to 20 Pa, 0.1 Pa to 10 Pa, 0.1 Pa to 1 Pa, 1 Pa to 1000 Pa, 1 Pa to 500 Pa, 1 Pa to 200 Pa, 1 Pa to 100 Pa, 1 Pa to 50 Pa, 1 Pa to 10 Pa, 10 Pa to 1000 Pa, 10 Pa to 500 Pa, 10 Pa to 200 Pa, 10 Pa to 100 Pa, 10 Pa to 50 Pa, 10 Pa to 20 Pa, 20 Pa to 1000 Pa, 20 Pa to 500 Pa, 20 Pa to 200 Pa, 20 Pa to 100 Pa, 20 Pa to 50 Pa, 50 Pa to 1000 Pa, 50 Pa to 500 Pa, 50 Pa to 200 Pa, 50 Pa to 100 Pa, 100 Pa to 500 Pa, 100 Pa to 200 Pa, 200 Pa to 1000 Pa, 200 Pa to 500 Pa, or 500 Pa to 1000 Pa. The yield stress can be measured, for example, using a stress ramp or stress sweep (e.g., from 1 Pa to 100 Pa, or from 1 Pa to 1000 Pa) in a parallel plate rheometer at a temperature of 25° C. to identify the stress at which the hydrogel exhibits a drop in viscosity.

The tan delta of the dynamic hydrogel (the ratio of the loss modulus (G″) over the storage modulus (G′)(tan(δ)=G″/G′)) can describe the overall viscoelasticity of the hydrogel (e.g., lower tan delta values correspond to more solid-like behavior, higher tan delta values correspond to more liquid-like behavior), and can correlate to the degradation rate of the hydrogel. In some embodiments, the dynamic hydrogels herein have a tan delta less than or equal to 1, 0.9, 0.8, 0.7, 0.6, 0.5, 0.4, 0.3, 0.2, or 0.1. The tan delta can be within a range from 0.1 to 1, 0.1 to 0.5, 0.1 to 0.3, 0.2 to 1, 0.2 to 0.5, or 0.5 to 1. The tan delta can be measured, for example, using an oscillatory shear test in a parallel plate rheometer at an angular frequency of 10 rad/s, a strain within the linear viscoelastic region of the hydrogel (e.g., 1% strain), and a temperature of 25° C.

In some embodiments, the dynamic hydrogels herein exhibit shear-thinning behavior, in that the viscosity of the dynamic hydrogel decreases with increasing shear rate and/or shear stress. Shear-thinning behavior can be advantageous, for example, to allow the dynamic hydrogel to be administered via injection. In some embodiments, the viscosity of the gel decreases with increasing shear rate at a shear rate within a range from 0.1 s⁻¹ to 1000 s⁻¹, for example, as observed on an oscillatory rheometer (e.g., a parallel plate rheometer) at 25° C. In some embodiments, the dynamic hydrogels herein have a viscosity within a range from 10 mPa-s to 2000 mPa-s, 10 mPa-s to 1000 mPa-s, 10 mPa-s to 500 mPa-s, 10 mPa-s to 200 mPa-s, 10 mPa-s to 100 mPa-s, 10 mPa-s to 50 mPa-s, 50 mPa-s to 2000 mPa-s, 50 mPa-s to 1000 mPa-s, 50 mPa-s to 500 mPa-s, 50 mPa-s to 200 mPa-s, 50 mPa-s to 100 mPa-s, 100 mPa-s to 2000 mPa-s, 100 mPa-s to 1000 mPa-s, 100 mPa-s to 500 mPa-s, 100 mPa-s to 200 mPa-s, 200 mPa-s to 2000 mPa-s, 200 mPa-s to 1000 mPa-s, 200 mPa-s to 500 mPa-s, 500 mPa-s to 2000 mPa-s, 500 mPa-s to 1000 mPa-s, or 1000 mPa-s to 2000 mPa-s at a shear rate of 1000 s⁻¹. The viscosity can be less than 10,000 mPa-s, 1000 mPa-s, or 100 mPa-s at a shear rate of 1000 s⁻¹. The viscosity can be measured, for example, using steady shear measurements in a parallel plate rheometer at a temperature of 25° C.

In some embodiments, the dynamic hydrogels herein exhibit self-healing behavior. Self-healing may refer to a process in which a gel that exhibits reduced resistance to flow when subjected to an external stress regains some or all of its rigidity and/or strength after the external stress is removed. Self-healing behavior can be advantageous, for example, to allow the dynamic hydrogel to form a cohesive depot after administration via injection and/or to limit burst release. In some embodiments, the dynamic hydrogels herein stop flowing and recover their mechanical properties in no more than 5 seconds, 10 seconds, 30 seconds, 1 minute, 2 minutes, 3 minutes, 5 minutes, or 10 minutes after the external stress is removed. Optionally, the modulus and/or viscosity of the dynamic hydrogel can recover to at least 90% of the initial value before application of the external stress within 5 minutes in a step-strain measurement (conducted with strains of 0.5% and 500%) or step-shear measurement (conducted with shear rates of 0.1 s⁻¹ and 100-1), respectively, on an oscillatory rheometer.

In some embodiments, the dynamic hydrogels herein exhibit viscoelastic behavior, in that the storage modulus (G′) of the hydrogel is dominant over the loss modulus (G″) at some point, for example, as observed in an oscillatory frequency sweep measurement in a range from 0.1 rad/s to 100 rad/s on an oscillatory rheometer performed in the linear viscoelastic region, yet the hydrogel exhibits complete stress relaxation following application of a constant strain of 500% within 15 minutes.

In some embodiments, the dynamic hydrogels described herein are biocompatible. A biocompatible material can be a material that is, along with any metabolites or degradation products thereof, generally non-toxic to the subject, and do not cause any significant adverse effects to the subject, at concentrations resulting from the degradation of the administered materials. A biocompatible material can be a material that does not elicit a significant inflammatory or immune response when administered to a subject.

In some embodiments, the dynamic hydrogels described herein are biodegradable. A biodegradable material can be a material that will degrade or erode under physiological conditions to smaller units or chemical species that are capable of being metabolized, eliminated, or excreted by the subject. For example, upon in vivo administration to a subject, the dynamic hydrogel can dissolve as the non-covalent bonds dissociate. The degradation rate of the dynamic hydrogel can be varied as desired, e.g., depending on the desired release profile for the therapeutic cargo. In some embodiments, following in vivo administration, the dynamic hydrogels are designed to persist at the administration site (e.g., remain as a cohesive depot) for at least 1 day, 2 days, 3 days, 4 days, 5 days, 6 days, 7 days, 8 days, 9 days, 10 days, 11 days, 12 days, 13 days, 14 days, 21 days, 28 days, 1 month, 2 months, 3 months, 4 months, 5 months, 6 months, 9 months, or 12 months. Alternatively or in combination, the dynamic hydrogels herein can persist at the administration site for no more than 12 months, 9 months, 6 months, 5 months, 4 months, 3 months, 2 months, 1 month, 28 days, 21 days, 14 days, 13 days, 12 days, 11 days, 10 days, 9 days, 8 days, 7 days, 6 days, 5 days, 4 days, 3 days, 2 days, or 1 day.

### II. Compositions for Delivery of Ocular Therapeutics and Associated Methods

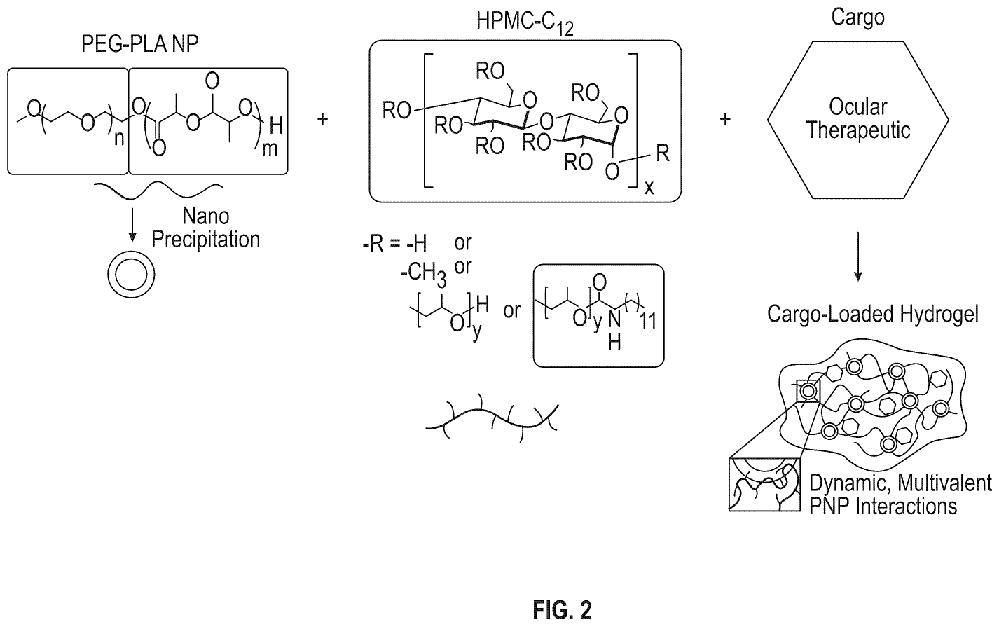

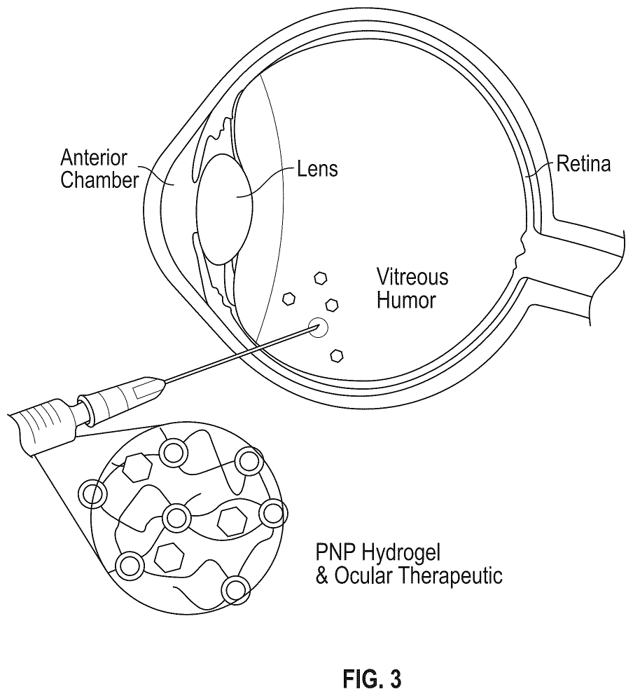

In some embodiments, the present technology provides compositions for delivery of ocular therapeutics for treating a disease or condition in a subject. The composition can include a dynamic hydrogel and at least one ocular therapeutic encapsulated by the dynamic hydrogel. The dynamic hydrogel can encapsulate the ocular therapeutic and provide sustained, controlled release of the ocular therapeutic when the composition is administered to an eye of a subject. In some embodiments, the dynamic hydrogel exhibits shear-thinning behavior that allows for facile administration via injection, as well as self-healing behavior that allows for formation of a cohesive depot that delivers the ocular therapeutic over a prolonged treatment period. For example, FIG. 2 is a schematic illustration of a PNP hydrogel prepared by mixing of hydrophobically-modified HPMC with PEG-PLA nanoparticles that allows for facile encapsulation of an ocular therapeutic, and FIG. 3 is a schematic illustration of formation of a localized depot in the VH following intravitreal injection of the PNP hydrogel, thus providing a tunable platform for sustained release of the ocular therapeutic.

In some embodiments, the ocular therapeutic comprises a drug configured to treat an eye disease, such as such as glaucoma, macular degeneration, cataract postoperative inflammation, uveitis, retinal vein occlusion, retinal artery occlusion, diabetic retinopathy, retinal phlebitis, proliferative vitroretinopathy, choroidal neovascularization, cystoid macular edema, age-related macular degeneration (e.g., dry age-related macular degeneration, wet age-related macular degeneration), vitreous macular adhesion, macular hole, optic neuritis, optic disc edema, optic nerve meningioma, optic nerve gliomas, retinoblastoma, and/or choroidoblastoma. For example, the ocular therapeutic can comprise a drug and may be any of the following or their equivalents, derivatives, or analogs, including prostaglandin analogs (PGAs) such as bimatoprost, latanoprost, travoprost, tafluprost, etc.; cholinergic agonists such as pilocarpine; anticholinergics such as atropine, scopolamine, etc.; beta blockers such as bunolol, metipranolol, propranolol, timolol, betaxolol, levobunolol, atenolol, befunolol, metoprolol, etc.; carbonic anhydrase inhibitors such as acetazolamide, dorzolamide, brinzolamide, methazolamide, dichlorphenamide, diamox, etc.; alpha adrenergic agonists such as apraclonidine, brimonidine, dipivefrine, etc.; antihypertensives such as guanethidine; alpha adrenergic blocker such as dapiprazole, and/or others. In some cases, the ocular therapeutic may comprise one or more biologics, such as one or more anti-vascular endothelial growth factor therapy (anti-VEGF) agents (e.g., pegaptanib sodium, ranibizumab, aflibercept, brolucizumab, faricimab, and/or others).

As mentioned, the ocular therapeutic can be a member of the PGA class of IOP-lowering medications. IOP is modulated in the anterior segment of the eye by balancing the production of aqueous humor by the ciliary body epithelium and its drainage through two outflow pathways: the conventional trabecular and unconventional uveoscleral pathways. Of the two pathways, the conventional trabecular meshwork pathway is specifically associated with increased flow resistance in glaucoma. PGAs reduce IOP by enhancing aqueous humor outflow. While it is better understood how PGAs modify outflow through the uveoscleral pathway, evidence suggests that PGAs may also act on the trabecular pathway by promoting changes in the extracellular matrix, leading to tissue remodeling through the regulation of matrix metalloproteinases (MMPs).

Optionally, the compositions herein can include other therapeutic cargo carried by the dynamic hydrogel, in addition to the ocular therapeutic. The other therapeutic cargo can include one or more therapeutic agents that produce a desired therapeutic effect, such as other small molecule drugs, peptides, proteins, polysaccharides, nucleic acids, cells, or combinations thereof. In some embodiments, the therapeutic agent(s) act in concert with the ocular therapeutic to treat an eye disease. Examples of such therapeutic agents include one or more anti-inflammatory drugs (e.g., dexamethasone, dexamethasone acetate, prednisone, prednisone acetate, fluocinolone, fluocinolone acetate, triamcinolone, methylprednisolone, methylprednisolone aceponate, halobetasol propionate, cortisone, hydrocortisone, and/or others), one or more immunosuppressive agents (e.g., cyclosporine, rapamycin, tacrolimus, mycophenolate mofetil, fujimycin, mizoribine, sulfasalazine, azathioprine, methotrexate and/or others), and/or other therapeutic agents. Such therapeutic agents can be encapsulated in the dynamic hydrogel via physical entrapment, interactions with hydrogel components (e.g., hydrophobic interactions), or suitable combinations thereof. Optionally, the therapeutic agent(s) can be administered to the subject separately from the ocular therapeutic via any suitable administration route (e.g., ITV or non-ITV administration).

In some embodiments, the present technology provides compositions including a dynamic hydrogel and one or more ocular therapeutics encapsulated by the dynamic hydrogel. The dynamic hydrogel carrying the ocular therapeutic can be any of the dynamic hydrogels described in Section I above. For example, the dynamic hydrogel can be a PNP hydrogel composed of a polymer and a plurality of nanoparticles that interact non-covalently with each other, as previously discussed in Section I.A. The dynamic hydrogel can exhibit shear-thinning, self-healing, and/or viscoelastic properties resulting from non-covalent, supramolecular interactions between the hydrogel components, as described above in Section I.B.

The composition can include any suitable amount of the ocular therapeutic for providing the desired therapeutic effect. For example, at least when the ocular therapeutic comprises a prostaglandin, the composition can include at least 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, or 30 μg of the ocular therapeutic. Alternatively or in combination, the composition can include no more than 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, 15 μg, 14 μg, 13 μg, 12 μg, 11 μg, 10 μg, 9 μg, 8 μg, 7 μg, 6 μg, or 5 μg of the ocular therapeutic. The amount of the ocular therapeutic in the composition can be within a range from 1 μg to 30 μg, 1 μg to 25 μg, 1 μg to 20 μg, 1 μg to 15 μg, 5 μg to 30 μg, 5 μg to 25 μg, 5 μg to 20 μg, 10 μg to 20 μg, or 15 μg to 25 μg. In those embodiments where the ocular therapeutic includes a biologic, such as an anti-VEGF agent, the composition can include at least 0.1 mg, 0.2 mg, 0.3 mg, 0.4 mg, 0.5 mg, 0.6 mg, 0.7 mg, 0.8 mg, 0.9 mg, or 1 mg of the biologic. Alternatively or in combination, the amount of the biologic in such compositions can include 0.1 mg to 1 mg, 0.2 mg to 0.9 mg, 0.3 mg to 0.8 mg, or 0.4 mg to 0.6 mg.

In some embodiments, the ocular therapeutic is present in the composition at a concentration of at least 0.05 mg/mL, 0.1 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, 0.19 mg/mL, 0.20 mg/mL, 0.25 mg/mL, 0.30 mg/mL, 0.35 mg/mL, 0.40 mg/mL, 0.45 mg/mL, 0.50 mg/mL, 0.55 mg/mL, 0.60 mg/mL, 0.65 mg/mL, 0.70 mg/mL, 0.75 mg/mL, 0.76 mg/mL, 0.80 mg/mL, 0.90 mg/mL, 1 mg/mL, 1.1 mg/mL, 1.2 mg/mL, 1.3 mg/mL, 1.4 mg/mL, 1.5 mg/mL, or 2 mg/mL. Alternatively or in combination, the concentration of the ocular therapeutic in the composition is no more than 2 mg/mL, 1.5 mg/mL, 1.0 mg/mL, 0.9 mg/mL, 0.8 mg/mL, 0.7 mg/mL, 0.6 mg/mL, 0.5 mg/mL, 0.4 mg/mL, 0.3 mg/mL, 0.2 mg/mL, or 0.1 mg/mL. The concentration of the ocular therapeutic in the composition can be within a range from 0.05 mg/mL to 2 mg/mL, 0.1 mg/mL to 1.5 mg/mL, 0.1 mg/mL to 1 mg/mL, or 0.1 mg/mL to 0.8 mg/mL.

In some embodiments, the size of the ocular therapeutic is smaller than the mesh size of the dynamic hydrogel. For example, the mesh size of the dynamic hydrogel can be greater than or equal to 2 nm, 2.5 nm, 3 nm, 3.5 nm, 4 nm, 4.5 nm, or 5 nm; while the size of the ocular therapeutic (e.g., hydrodynamic diameter) can be less than 2 nm, 1.5 nm, 1 nm, or 0.5 nm. Accordingly, physical entrapment of the ocular therapeutic by the hydrogel network may be ineffective for producing controlled release if the therapeutic is sufficiently hydrophilic and non-interacting with the hydrogel structure, in that the ocular therapeutic may be able to diffuse freely out of the dynamic hydrogel, resulting in uncontrolled burst release in vivo. For example, physical entrapment may be insufficient for controlling the release of an ocular therapeutic having a molecular weight less than or equal to 10 kDa

In such embodiments, the dynamic hydrogel can include at least one component that binds to the ocular therapeutic to control the release of the ocular therapeutic from the dynamic hydrogel. The interaction can be a non-covalent interaction, such as a hydrophobic interaction. For instance, in embodiments where the ocular therapeutic is a hydrophobic molecule (e.g., bimatoprost) or includes a hydrophobic moiety (e.g., a lipophilic substituent such as a fatty acid side chain), the ocular therapeutic can interact with one or more hydrophobic components of the dynamic hydrogel (e.g., for a PNP hydrogel, the hydrophobic surfaces of the nanoparticles and/or hydrophobic moieties on the polymer chain). These hydrophobic interactions can cause the ocular therapeutic to adhere to the hydrophobic components of the dynamic hydrogel, thus inhibiting uncontrolled diffusion of the therapeutic peptide out of the dynamic hydrogel. In such embodiments, the therapeutic peptide can be released from the dynamic hydrogel primarily or entirely via erosion of the dynamic hydrogel in vivo. The release kinetics of the ocular therapeutic can thus be adjusted by tuning the degradation rate of the dynamic hydrogel (e.g., by controlling the storage modulus of the dynamic hydrogel).

In other embodiments, however, the size of the ocular therapeutic can be larger than the mesh size of the dynamic hydrogel (e.g., if the ocular therapeutic is a biologic such as an antibody). In such embodiments, the ocular therapeutic can be encapsulated in the dynamic hydrogel via physical entrapment by the hydrogel network.

As previously mentioned, the PNP hydrogels described herein can be formed through dynamic interactions between hydrophobically-modified polymers and nanoparticles, such as dodecyl-modified HPMC (HPMC-C₁₂) and biodegradable polymeric nanoparticles composed of PEG-PLA. The polymers can bridge between nanoparticles and dynamically interact with the nanoparticle surfaces. The lipid tails of the polymers may also bind to retinal ganglion cells lining the inner surface of the vitreous chamber, which may cause damage to the cells (e.g., via infiltration into and/or disruption of cell membranes). To reduce such interactions, the polymer of the PNP hydrogels can be modified with hydrophobic moieties that form relatively shorter lipid tails that are less likely to mimic cellular lipids, and thus less likely to bind with the retinal ganglion cells. The hydrophobic moiety used for the modification, for example, can comprise fewer than 12 carbon atoms, such as butyl (—C4), hexyl (—C6), octyl (—C8), and decyl (—C10). When using hydrophobic moieties with fewer carbon atoms, it can be beneficial to maintain the total mass (wt %) of hydrophobic moieties in the polymer in order to maintain a hydrophobicity that will retain the structure of the PNP hydrogel. This can be accomplished by increasing the molar ratio of the hydrophobic moiety to the polymer to compensate for the decreased molecular weight of the hydrophobic moiety. For example, if a PNP hydrogel comprising a dodecyl-modified HPMC (HPMC-C₁₂) contains 10 wt % dodecyl in the polymer and is prepared using a molar ratio of dodecyl to HPMC of X, then a PNP hydrogel comprising a hexyl-modified HPMC (HPMC-C6) with 10 wt % hexyl in the polymer can be prepared using a molar ratio of hexyl to HPMC of 2X (since hexyl has approximately half the molecular weight of dodecyl).

In some instances, PNP hydrogels formulated with smaller particles (e.g., particles having a diameter within a range from 25 nm to 30 nm) may produce toxicity due to direct uptake of the smaller particles by the cells. To reduce these effects, the PNP hydrogels herein can be formulated with larger particles, such as particles having a diameter of at least 40 nm, 50 nm, 60 nm, 70 nm, 80 nm, 90 nm, or 100 nm; and/or a diameter within a range from 50 nm to 100 nm, or 70 nm to 90 nm.

It can be desirable for the fluid and/or material properties of the dynamic hydrogels to match the fluid and/or material properties of the VH. For example, in some cases the dynamic hydrogels can have a density such that the dynamic hydrogels are configured to reside away from the wall of the vitreous chamber, rather than sink to the bottom or rise to the top. Moreover, dynamic hydrogels such as PNP hydrogels can have mechanical characteristics that are generally similar to the mechanical characteristics of the VH (e.g., a storage modulus on the order of a few hundred Pa), whereas conventional covalently crosslinked gels are generally significantly stiffer (e.g., a storage modulus on the order of several kPa). Additionally or alternatively, the polymers used in the PNP hydrogels can be tuned to avoid large fluctuations in VH pH during degradation of the polymer. In some cases, for instance, it may be advantageous to use polycaprolactone (PCL) in place of PGA (thus forming PEG-PCL), as PCL has a slower degradation rate and is less likely to erode (and release acid) within the hydrogel. Alternatively or in combination, the PNP hydrogels can be configured to degrade over longer time frames to reduce the rate of acid release. As described herein, PNP hydrogels having a higher storage modulus may be stiffer and degrade more slowly than gels having a lower storage modulus.

In some embodiments, the composition of the degradable polymer block in the nanoparticles may be modified to reduce the potential of a foreign body response (e.g., via reduction of immune cell activation and/or toxicity to retinal cells). For example, the hydrophilic portion of the nanoparticle copolymer blend may comprise an alternative to PEG, such as a polyacrylamide, a polyoxazoline, etc. Likewise, the hydrophobic portion of the nanoparticle blend may comprise an alternative to PLA, such as PCL, PHB, etc. The nanoparticles can comprise a PEG-alternative mixed with PLA, a PEG-alternative mixed with a PLA-alternative, PEG mixed with a PLA-alternative, or another combination of polymers.

Additionally or alternatively, the cellulose polymer backbone may be modified to reduce the potential of a foreign body response (e.g., via reduction of immune cell activation and/or toxicity to retinal cells). The cellulose polymer backbone may comprise, for example, other cellulosic polymers such as methylcellulose or carboxymethylcellulose. In some embodiments, the hydrogel polymer could comprise hyaluronic acid (HA).

The compositions herein can be administered to the subject via any suitable route, such as ITV injection. The shear-thinning properties of the dynamic hydrogel can allow for delivery via injection, while the self-healing properties of the dynamic hydrogel can allow for formation of a depot at the injection site that produces controlled release of the ocular therapeutic over the desired treatment period. Injection of the composition can be performed using any suitable tubular device having a lumen configured for delivery of a hydrogel, such as needles (e.g., hypodermic needles, surgical needles, infusion needles), injector pens, catheters, trocars, cannulas, tubing, etc. For instance, the composition can be delivered through a 27-gauge, 28-gauge, 29-gauge, or 30-gauge needle. The composition can be formulated to have a volume that is sufficiently small for injection into the VH without significant increases in IOP, such as a volume less than or equal to 1 mL, 0.5 mL, 0.25 mL, 0.1 mL, 0.075 mL, 0.05 mL, 0.025 mL, 0.015 mL, or 0.01 mL. In some embodiments, the composition is administered as a single injection at a single injection site of the eye, while in other embodiments, the composition can be administered as multiple injections at the same or different injection sites of the eye. For example, in some cases multiple injections can be made in the same eye, each having the same or different amounts of the ocular therapeutic. The use of multiple injections can be beneficial for tailoring the dosage from subject to subject. The composition can be administered to the subject at any suitable frequency, such as once per week, once per 2 weeks, once per 4 weeks, once per month, once per 2 months, once per 3 months, once per 4 months, once per 5 months, once per 6 months, once 9 months, or once per year.

The compositions herein can be configured to deliver a therapeutically effective amount of the ocular therapeutic over a desired treatment period, which can be an amount that is effective to ameliorate or prevent a symptom of a disease or condition in a subject. For example, the treatment period can be at least 1 day, 2 days, 3 days, 4 days, 5 days, 6 days, 7 days, 8 days, 9 days, 10 days, 11 days, 12 days, 13 days, 14 days, 28 days, 35 days, 40 days, 50 days, 60 days, 70 days, 80 days, 90 days, 100 days, 110 days, 120 days, 150 days, 180 days, 6 months, 7 months, 8 months, 9 months, 10 months, 11 months, 12 months, or 1 year. The treatment period can be approximately 2 weeks, 4 weeks, 1 month, 2 months, 3 months, 4 months, 5 months, 6 months, 9 months, or 12 months. The treatment period can be modulated by tuning the degradation rate of the hydrogel, as described elsewhere herein.

In some embodiments, the present technology provides methods for treating an ocular condition of a subject by administering a composition as described herein. The composition can treat a disease or condition of the subject by producing a desired therapeutic effect in the subject, such as alleviation of symptoms, a reduction in the severity of the disease or condition, inhibiting an underlying cause of the disease or condition, steadying the disease or condition in a non-advanced state, delaying the progress of a disease or condition, and/or improvement or alleviation of the disease or condition. For example, the compositions herein can be configured to treat glaucoma by reducing a mean IOP (relative to pre-injection baseline) of the subject by an amount between 7 and 10 mmHg, or between 8 and 9 mmHg. Examples of other diseases and/or conditions of the eye that may be treated using the compositions described herein include macular degeneration, cataract postoperative inflammation, uveitis, retinal vein occlusion, retinal artery occlusion, diabetic retinopathy, retinal phlebitis, proliferative vitroretinopathy, choroidal neovascularization, cystoid macular edema, age-related macular degeneration, vitreous macular adhesion, macular hole, optic neuritis, optic disc edema, optic nerve meningioma, optic nerve gliomas, retinoblastoma, and/or choroidoblastoma, and others.

In some embodiments, the present technology provides methods for preparing a composition for treating a disease or condition as described herein. The method can include combining the components of a dynamic hydrogel (e.g., polymer and nanoparticles) with one or more ocular therapeutics (e.g., a PGA such as bimatoprost), thus forming a dynamic hydrogel encapsulating the ocular therapeutic. The combining of the hydrogel components and ocular therapeutic can be performed using simple mixing under gentle conditions, such as physiological pH (e.g., pH 7.0 to 7.4) at room temperature (e.g., 25° C.) or physiological temperature (e.g., 37° C.). Optionally, the method can include combining the hydrogel components and ocular therapeutic with other components, such as an additional therapeutic agent (such as an anti-inflammatory agent, an immunosuppressive agent, etc.). In some embodiments, the composition is prepared no more than 1 hour, 30 minutes, 15 minutes, 10 minutes, 5 minutes, 2 minutes, or 1 minute before administering the composition to the subject. Alternatively or in combination, the composition can be prepared at least 1 minute, 2 minutes, 5 minutes, 10 minutes, 15 minutes, 30 minutes, or 1 hour before administering the composition to the subject. The dynamic hydrogel can be mostly or fully formed before the composition is administered to the subject. For example, the dynamic hydrogel can be sufficiently crosslinked (e.g., non-covalently crosslinked) to exhibit the shear-thinning, self-healing, and/or viscoelastic properties described herein before the composition is administered to the subject.

In some embodiments, the present technology provides kits for preparing a composition as described herein. The kit can include a solution containing an ocular therapeutic and one or more solutions containing the components of a dynamic hydrogel (e.g., a solution containing a polymer and a solution containing nanoparticles, or a single solution containing a polymer and nanoparticles). The solutions can be provided in tubes, bottles, ampoules, syringes, or any other suitable storage container. In some embodiments, the solutions each independently include a suitable pharmaceutically acceptable diluent. The pharmaceutically acceptable diluent can be any diluent that does not substantially produce adverse reactions, e.g., toxic, allergic, or immunological reactions, when administered to a subject. Examples of pharmaceutically acceptable diluents include, but are not limited to, saline, Ringer's solution, dextrose solution, phosphate buffered saline, water, or a combination thereof. The pharmaceutically acceptable diluent can include an isotonicity imparting agent, such as sodium chloride, potassium chloride, or monosodium phosphate. The pharmaceutically acceptable diluent can include a buffer, such as bicarbonate, TRIS, HEPES, MOPS, CHES, CHAPS, or phosphate buffered saline. The pharmaceutically acceptable diluent can include stabilizers and/or preservatives, as appropriate. Additional examples and details of pharmaceutically acceptable diluents can be found in Martin, Remington's Pharmaceutical Sciences, 21st Ed., Mack Publ. Co., Easton, Pa. (2005), which is incorporated herein by reference in its entirety.

### EXAMPLES

The present technology is further illustrated by the following non-limiting examples.

### Example 1: Preparation of a PNP-2-10 Hydrogel for Intravitreal Extended Drug Release

This example describes a process for preparing a PNP-2-10 hydrogel for intravitreal extended drug release. Hydroxypropylmethylcellulose (HPMC, meets USP testing specifications), N-methyl-2-pyrrolidone (NMP), 1-dodecylisocynate, N,N-diisopropylethylamine (Hunig's base), acetone, monomethoxy-PEG (5 kDa), diazobicylcoundecene (DBU), acetic acid, formic acid, diethyl ether, hexanes, dimethyl sulfoxide (DMSO), acetonitrile, albumin-FITC, agar, and fluorescein were purchased from Sigma-Aldrich and used as received. Dichloromethane (DCM) was purchased from Sigma-Aldrich and further dried via cryo distillation. Lactide (LA) was purchased from Sigma-Aldrich and purified by recrystallization in ethyl acetate with sodium sulfate. Sodium hyaluronate (HA, research grade, 1.0-1.8 MDa) was purchased from Lifecore Biomedical. Acetonitrile (HPLC grade; J.T. Baker) and MilliQ water were used for all HPLC analysis. Bimatoprost was purchased from Toronto Research Chemicals.

HPMC-C₁₂ was prepared as follows. HPMC (1.0 g) (SEC MALS: Mw (Ð)=372.4 kDa (1.43)) was dissolved in NMP (40 mL) at room temperature with stirring. Once the polymer had completely dissolved, the reaction was brought to 80° C. and a solution of 1-dodecylisocynate (0.5 mmol) in NMP (5 mL) was added dropwise, followed by Hunig's base (catalyst, ˜10 drops). The reaction was removed from heat and allowed to react with stirring at room temperature for 16 hours. The solution was then precipitated from acetone and hydrophobically-modified HPMC was recovered by dialysis against MilliQ water for 3-4 days (MWCO 3.5 kDa) and lyophilization, yielding HPMC-C₁₂ as a white amorphous powder. The polymer was reconstituted as a 60 mg mL⁻¹ solution with sterile PBS, pH 7.4, prior to use in hydrogels.

PEG-PLA was prepared and analyzed as follows. Recrystallized LA (10 g) was dissolved in cryo-distilled DCM (50 mL) under nitrogen with mild heating. Methoxy poly(ethylene glycol) (5 kDa; 2.5 g) was heated to 90° C. under vacuum for 30 minutes, allowed to cool slightly under nitrogen, and then dissolved in cryo-distilled DCM (5 mL) with distilled DBU (75 μL; 0.5 mmol; 0.7 mol % relative to LA). The PEG/DBU solution was added rapidly to the LA solution and allowed to stir for 8 min. The reaction mixture was quenched with acetone (500 μL) and acetic acid (˜2 drops) and precipitated from excess 50:50 mixture ethyl ether and hexanes. The PEG-PLA copolymer was collected and dried under vacuum to yield a white amorphous powder. According to gel permeation chromatography (GPC) using dimethylformamide (DMF) as the solvent, the PEG-PLA copolymer exhibited Mw (Ð)=22.5 kDa (1.07).

PEG-PLA nanoparticles (NPs) were prepared and analyzed as follows. A solution (1 mL) of PEG-PLA in 25:75 DMSO: Acetonitrile (50 mg mL⁻¹) was added dropwise to water (10 mL) at a stir rate of 600 rpm. NPs were purified by ultracentrifugation over a filter (MWCO 10 kDa; Millipore Amicon Ultra-15) followed by resuspension in PBS to a final concentration of 200 mg mL⁻¹. NP size and dispersity were characterized by DLS (Wyatt DynaPro PlateReader-II; average diameter=31.8 nm, PDI=0.04).

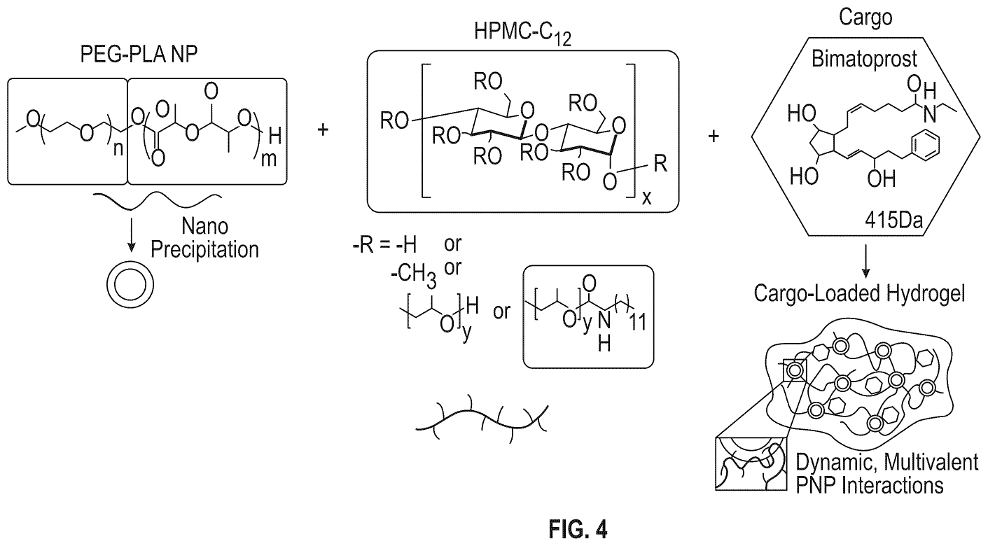

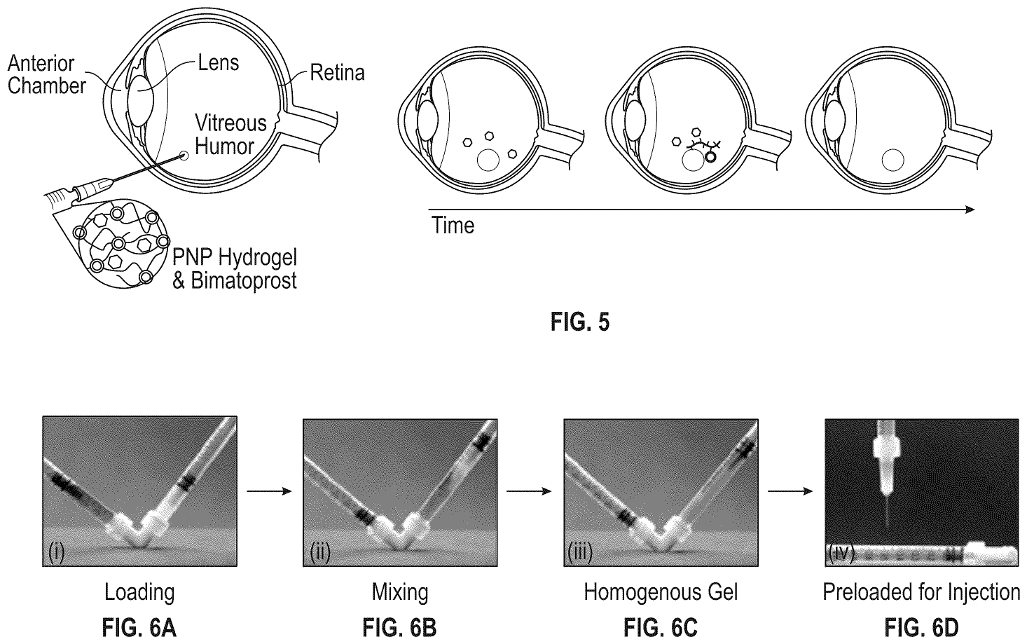

FIG. 4 is a schematic illustration of a PNP hydrogel prepared by mixing of hydrophobically-modified HPMC with PEG-PLA nanoparticles that allows for facile encapsulation of an ocular therapeutic, and FIG. 5 is a schematic illustration of formation of a localized depot in the VH following intravitreal injection of the PNP hydrogel, thus providing a tunable platform for sustained release of the ocular therapeutic. PNP-2-10 hydrogels were formulated with final concentrations of 2 wt % HPMC-C12 and 10 wt % PEG-PLA NPs. HPMC-C₁₂ was dissolved in PBS at 6 wt % and loaded into a luer-lock syringe (1 or 3 mL depending on volume of gel needed). A 20 wt % solution of NPs in PBS was diluted with additional PBS, or PBS containing bimatoprost at the desired concentration, and loaded into a separate luer-lock syringe (1 or 3 mL). As shown in FIGS. 6A-6D, the nanoparticle syringe was then connected to a female-female luer-lock elbow and the solution was moved into the elbow until visible at the other end. The HPMC-C12 syringe was then attached to the other end of the elbow with care to avoid air at the interface of HPMC-C₁₂ and the NP solution. The two solutions were mixed for 1 minute or until a homogenous PNP hydrogel was formed. After mixing, the elbow was removed and a needle of the appropriate gauge was attached.

### Example 2: Rheological Characterization of PNP-2-10 Hydrogels

This example describes rheological characterization of PNP-2-10 hydrogels with and without bimatoprost (0.25 mg mL⁻¹) with the goal of understanding whether bimatoprost interferes with the hydrogel's injectability and depot formation properties. The PNP-2-10 hydrogels were prepared as described in Example 1. Rheological characterization was performed using a TA Instruments DHR-2 stress-controlled rheometer. All experiments were performed using a 20 mm diameter serrated plate geometry at 25° C. with a 500 μm gap. Frequency sweep measurements were performed at a constant 1% strain in the linear viscoelastic regime. Stress sweeps were performed from low to high with steady state sensing and yield stress values extracted. Flow sweeps were performed from high to low shear rates. Step shear experiments were performed by alternating between a low shear rate (0.1 s⁻¹; 60 s) and a high shear rate (10 s⁻¹; 30 s) for three cycles.

For the injectability calculation and generation of an Ashby-style plot, high shear data (shear rates 0.1-10 rad s⁻¹) from flow sweep rheology was fit with the power law η=Kγⁿ^{^{−1}} relating viscosity (η) and shear rate (γ) in GraphPad Prism and values for consistency index (K) and shear-thinning parameter (n) extracted. Values were plotted as points (n, K) along with the

$${K}_{max} \leq ( \frac{{P}_{max} R}{2 L} ) {( \frac{π {R}^{3}}{{Q}_{min}} )}^{n} {( \frac{n}{3 n + 1} )}^{n}$$

where Pmax is the maximum pressure to be exerted during injection, R is the diameter of the needle, L is the needle length, and Qmin is the minimum desired flow rate. The following parameters were used: Pmax=2.6 MPa, R=133 μm, L=8.5 mm, and Qmin=6 mL min⁻¹ (50 μL in 0.5 seconds).

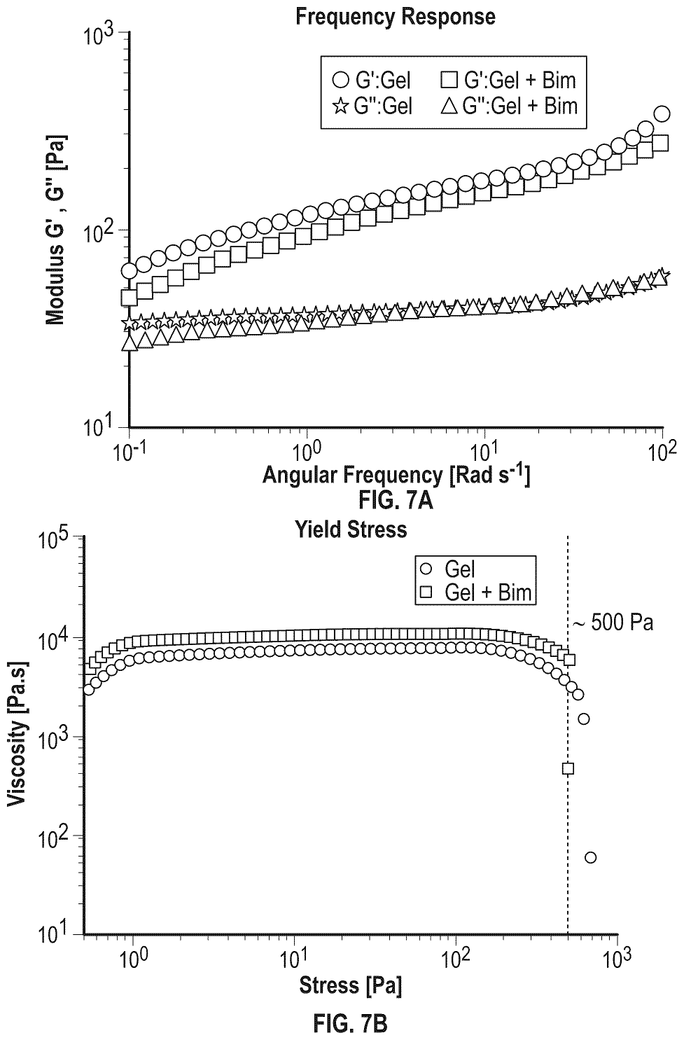

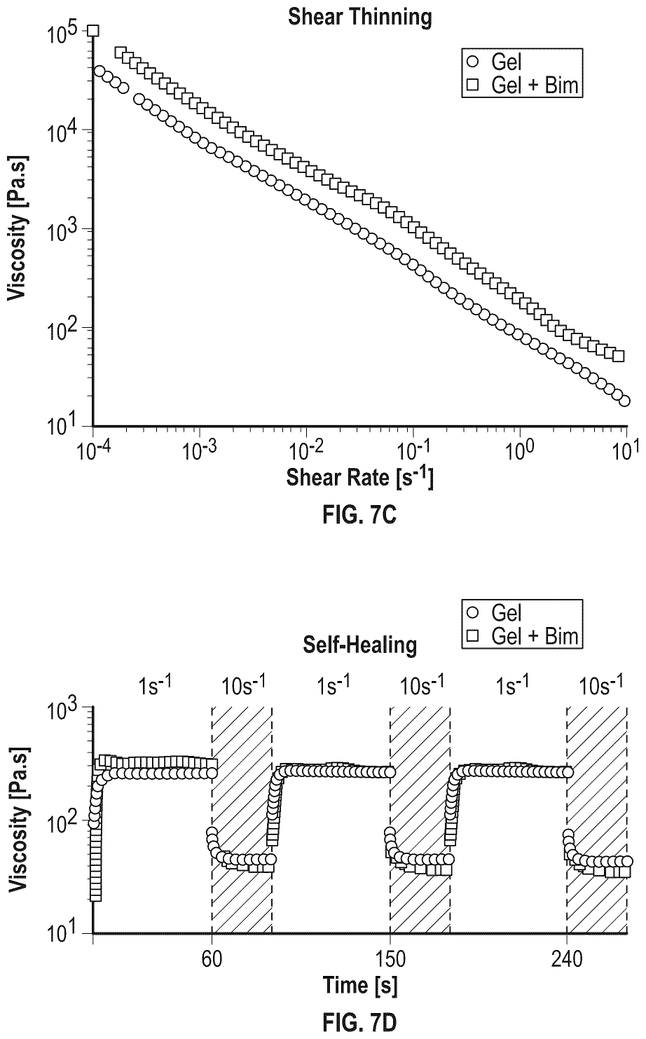

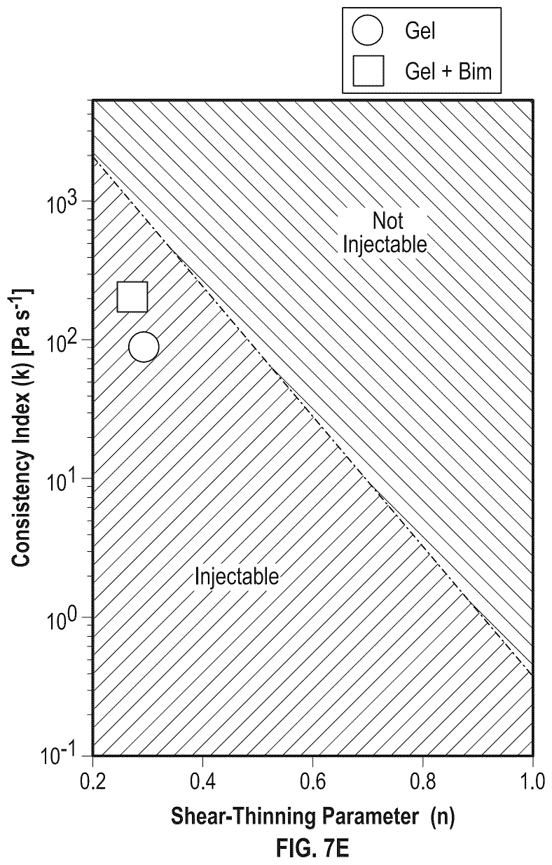

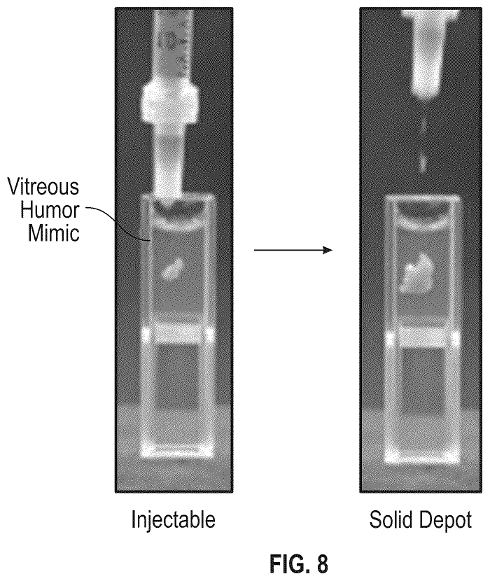

As depicted in FIG. 7A, frequency-dependent oscillatory shear rheology showed that the frequency responses of the materials were unchanged with encapsulation of bimatoprost. Indeed, both formulations showed robust solid-like properties across the entire range of timescales evaluated, evident from the storage modulus G′ being larger than the loss modulus G″. Stress-controlled flow sweep measurements revealed that both materials exhibit static yield stresses of approximately ˜500 Pa (FIG. 7B), and steady shear flow rheology demonstrated robust shear-thinning behavior with dramatically reduced viscosities observed at high shear rates (FIG. 7C). These characteristics are indicative of a material that will yield and shear-thin, enabling injection through a syringe and needle. It is also advantageous for this material to recover its mechanical properties rapidly following injection to limit drug burst release and form a robust depot for extended release. To assess the self-healing of the materials, consecutive periods of high (10 rad s⁻¹) and low (1 rad s⁻¹) stress were applied to the hydrogel formulations, as shown in FIG. 7D. Over several cycles, drops in viscosity were observed at high stress and rapid recovery of the material properties was observed at low stress, indicating the high stress disrupts the non-covalent PNP interactions responsible for crosslinking in these systems, and these interactions reform once the stress is removed. To further evaluate injectability of the PNP gels, the steady shear flow rheology data was fit with the power law η=Kγⁿ^{^{−1}}, which relates viscosity (η) and shear rate (γ) for non-Newtonian complex fluids, and values for the consistency index (K) and shear-thinning parameter (n) were extracted. These values fall within the region of “injectability” on an Ashby-style plot of K and n (FIG. 7E) to capture the injection forces that are applicable by healthcare professionals. Further, it was demonstrated that both empty and bimatoprost-loaded PNP hydrogels were easily injected through a 30-gauge needle, and rapidly formed a robust depot in a VH mimic (FIG. 8).

### Example 3: Evaluation of PNP-2-10 Hydrogel Administration of a Model Cargo in a VH Mimic

This example describes PNP-2-10 hydrogel depot formation and model cargo release in a VH mimic. The PNP-2-10 hydrogel was prepared as described in Example 1. The present technology is directed to ITV administration of PNP hydrogels in the sensitive environment of the eye. To better visualize and understand how the PNP hydrogels would behave in the VH, depot formation and model cargo release were analyzed in a VH mimic composed of agar and hyaluronic acid (HA). The VH mimic was 0.95 mg/mL agar and 0.7 mg/mL HA (1.01-1.8 MDa) in PBS, i.e. 0.095% Agar+0.07% HA, which was heated until the agar and HA fully dissolved, then pipetted into cuvettes, capped/sealed, and allowed to set overnight at room temperature before use. For comparison, PNP hydrogels containing either fluorescein (a model small molecule similar in size and hydrophobicity to bimatoprost) or albumin-FITC (a model protein) were administered through a 30-gauge needle into a cuvette containing the VH mimic. Bolus administrations of PBS formulations of each dye were used as controls to assess the prolonged delivery of these model molecules from the hydrogel depots.

To prepare the VH mimic release assay, 50 μL of PNP hydrogel or PBS loaded with either 0.25 mg mL⁻¹ fluorescein or 10 mg mL⁻¹ albumin-FITC (MW˜66 kDa, mol FITC: mol albumin=14) was injected through a 30-gauge needle into a cuvette containing a VH mimic. Cuvettes were imaged at 0, 0.25, 0.5, 1, 1.5, 2, 3, 4, 5, 6, 7, 25.5, and 75 hours with dark and light backgrounds for visualization of the dye and gel depot. Dye release was quantified using ImageJ software. Images were separated into red, blue, and green channels and the intensity in the green channel within a region inside the hydrogel depot or initial PBS injection site was measured for each time point, denoted Iₜₙ. A region in the lower right of the cuvette (outside of the injection site) was used for background intensity measurements to determine when each PBS sample had completely diffused, denoted tₚₗₐₜₑₐᵤ. The fraction of dye remaining in the gel depot at the final time point was determined by comparing the change in background intensity from times t₀ to tₚₗₐₜₑₐᵤ for PBS and gel samples:

$${Dye}_{remaining \cdot gel} = {Dye}_{r} = \frac{{\vert {I}_{{t}_{0}} - {I}_{{t}_{plateau}} \vert}_{gel}}{{\vert {I}_{{t}_{0}} - {I}_{{t}_{plateau}} \vert}_{gel}} .$$

The percent of dye retained in the gel or PBS injection site at each time point tn was determined by normalizing the intensity as

$$\% Dye {retained}_{n} = \frac{{I}_{{t}_{n}} - C * {I}_{{t}_{f}}}{{I}_{{t}_{0}} - C * {I}_{{t}_{f}}} ,$$

where t_{f} is the final time point measured and C is a constant defined by

$$C = \frac{{I}_{{t}_{f}} - {Dye}_{r} * {I}_{{t}_{0}}}{( 1 - {Dye}_{r} ) * {I}_{{t}_{f}}} .$$

Note, tor these samples, t_{f}=tₚₗₐₜₑₐᵤ.

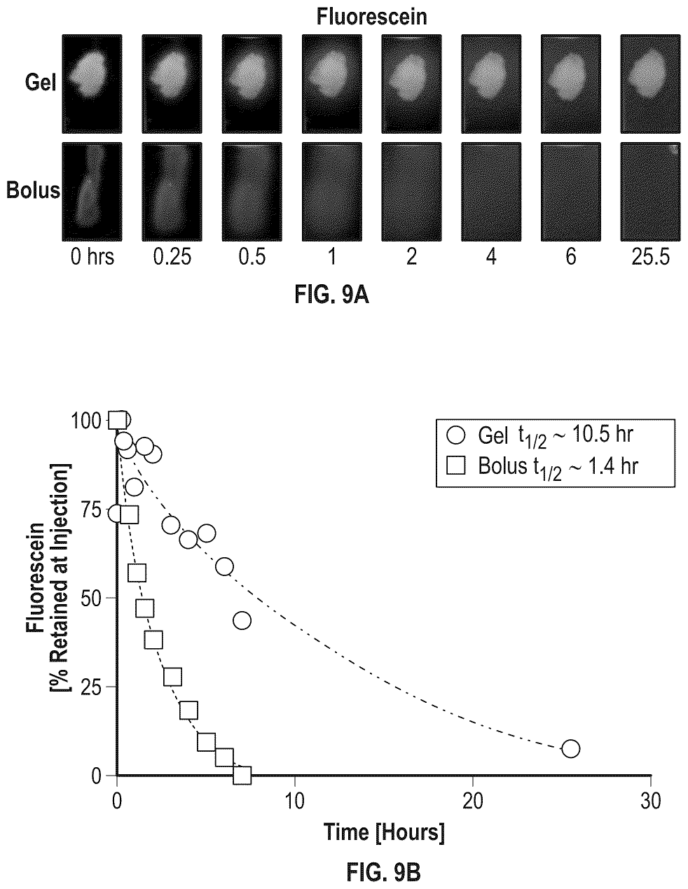

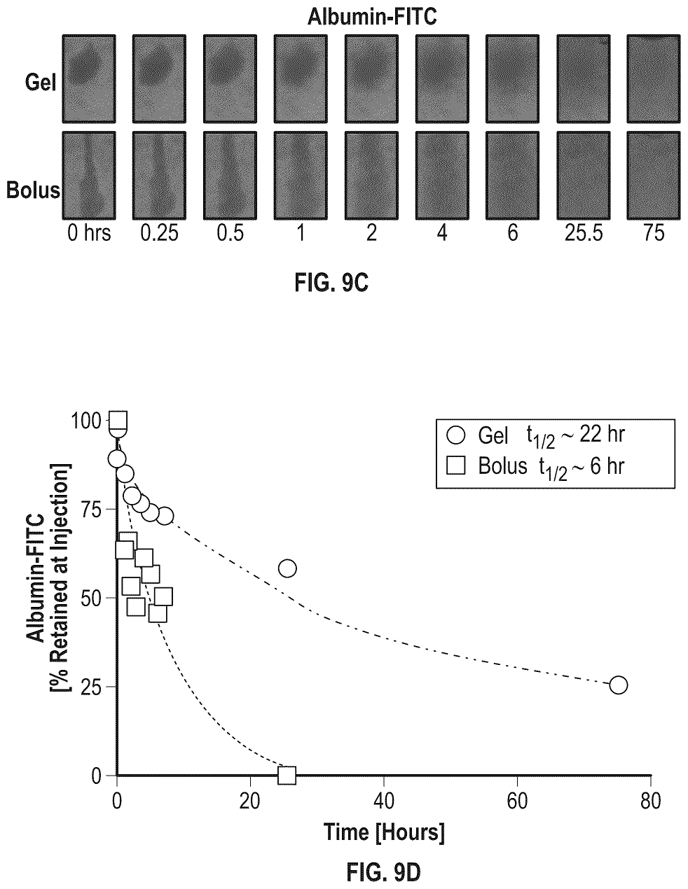

With reference to the images shown in FIGS. 9A and 9C, the cargo-loaded PNP hydrogels immediately formed depots locally retaining the cargo, whereas cargo administered in PBS boluses rapidly diffused from the injection site following the path of the needle. Over the course of the release experiment, the PNP hydrogel slowed release of both model cargos from the injection site, increasing the effective half-life of retention of fluorescein by over 7.5-fold and of albumin-FITC by over 3.7-fold compared to the control PBS bolus (see FIGS. 9B and 9D). Minor swelling of the PNP hydrogels was observed over time, and the depot remained suspended, not sinking or floating in the VH mimic, indicating that these materials should remain suspended when injected into the vitreous chamber of the eye rather than contacting the sensitive tissues (e.g. lens, ciliary body, or retina) of the eye.

### Example 4: In Vitro Release of Bimatoprost from PNP Hydrogels Under Static and Dynamic Conditions

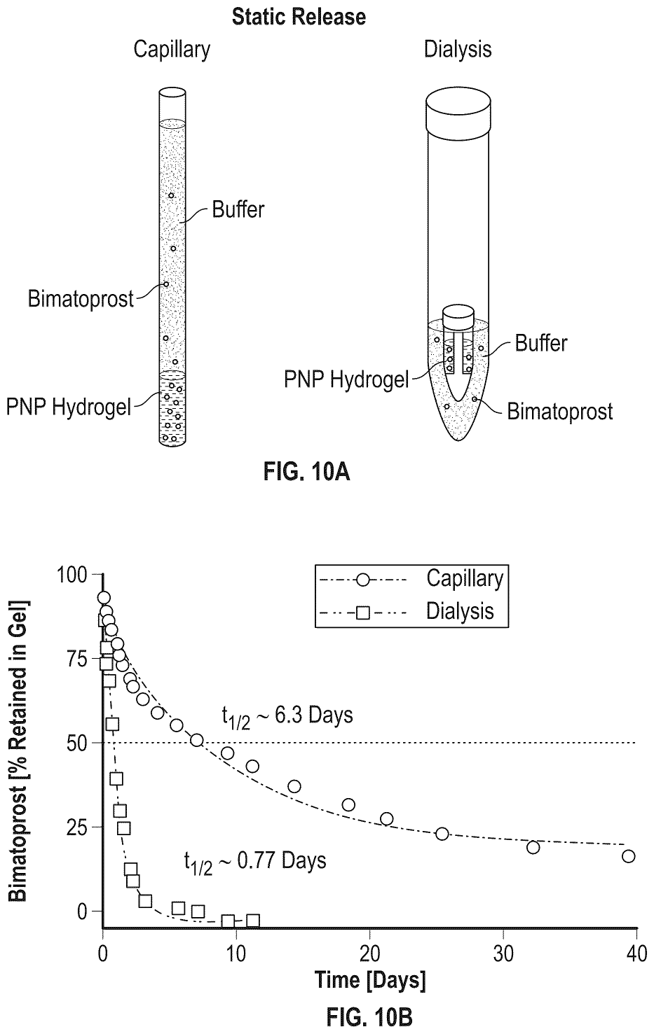

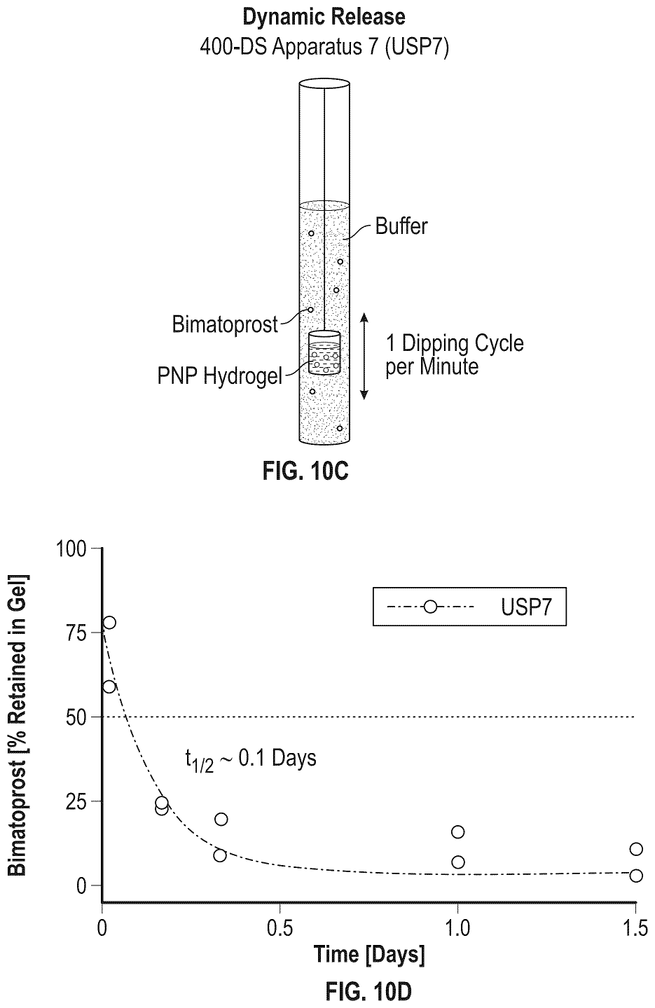

This example describes the in vitro release of bimatoprost from a PNP-2-10 hydrogel where the PNP-2-10 hydrogel was prepared as described in Example 1. To characterize the release behavior of bimatoprost from the PNP-2-10 hydrogels, infinite sink release assays were conducted in three separate configurations to later identify which release study formats better recapitulate physiological conditions. Two static assays (FIG. 10A) and one dynamic release assay (FIG. 10C) were implemented. The first static release assay employed a glass capillary tube, where the hydrogel was injected into the bottom of the tube and confined with a single surface in contact with release buffer (PBS). The release buffer was removed and replaced at each time point to maintain infinite sink conditions and to evaluate bimatoprost release over time. Without being bound by theory, it was hypothesized that this first configuration would mimic hydrogel injected into a treatment area surrounded with little biological fluid and minimal tissue motion, such as the subcutaneous space, and a good correlation between in vitro and in vivo release characteristics in this configuration has been previously demonstrated. The second assay employed a static dialysis release setup, whereby hydrogel was injected into a microcentrifuge tube with dialysis membrane walls and placed in a sealed tube containing release buffer (PBS). At each time point, a small proportion of the release buffer was removed (˜5% total volume) for bimatoprost quantification and replaced with fresh buffer. Without being bound by theory, it was hypothesized that this second configuration would better mimic hydrogel administered into an area with extensive exposure to fluid without significant motion, such as VH.

The in vitro static capillary and dialysis release assays were performed as follows. For capillary release, glass capillary tubes were sealed on one end with epoxy and allowed to cure for at least 24 hours. PNP hydrogel loaded with bimatoprost (100 μL, 0.25 mg mL⁻¹) was injected into the bottom of each tube (n=3) and PBS (400 μL) was injected on top carefully to not disrupt the gel surface. Tubes were sealed with parafilm and stored upright at 37° C. At each time point, all 400 μL PBS was carefully removed from the tube and replaced with fresh PBS, avoiding disturbance of the gel surface. Samples were taken at: 2, 6, 9, 13, 23, 29, 36, 48, 54, 72 hours and 4, 5, 7, 9, 11, 14, 18, 21, 25, 32, 39 days.

For dialysis release, PNP hydrogel loaded with bimatoprost (100 μL, 0.25 mg mL⁻¹) was injected into mini dialysis tubes (molecular weight cutoff of 12-14 kDa; Sigma-Aldrich Pur-A-Lyzer). Each tube (n=3) was sealed with parafilm, placed in PBS (5 mL) in a sealed 15 mL Falcon tube, and stored upright at 37° C. At each time point, 250 μL of PBS was removed and replaced with an equal volume of fresh PBS. Samples were taken at: 2, 6, 9, 13, 23, 29, 36, 48, 54, 72 hours and 4, 5, 7, 9, 11 days.

At the end of the study, gel was collected from each capillary or dialysis tube, diluted with PBS and remaining bimatoprost was quantified. Bimatoprost concentration in releasate was quantified using HPLC-MS. Data is presented as bimatoprost remaining in gel, calculated as

$$1 - \frac{{M}_{t}}{{M}_{inf}} ,$$

where Mₜ is the amount released at each time point and M_{inf} is the total amount loaded in the gel at the beginning of the assay. Data were fit with a one phase-decay in GraphPad Prism and the half-life of release was determined. Ritger-Peppas analysis was performed by fitting data with power law

$$\frac{{M}_{t}}{{M}_{inf}} = {kt}^{n}$$

in GraphPad Prism to find k and n.

Release profiles from both static assays showed no evidence of burst release, which is a key advantage of the PNP hydrogel over other injectable hydrogel systems. The capillary configuration yielded a half-life of release of 6.3 days, which was much longer than the half-life observed in the dialysis configuration, which was only 0.77 days (FIG. 10B). This observation suggests that extensive fluid exposure can lead to faster-than-expected erosion of the PNP hydrogels. Cargo release from polymeric materials can be described by the exponential relationship derived by Ritger and Peppas:

$$\frac{{M}_{t}}{{M}_{∞}} = {kt}^{n}$$

where the release constant (k) and diffusional exponent (n) are characteristic of the release mechanism. Pure Fickian diffusion yields an n value of 0.43 for a sphere, whereas values above this indicate some degree of anomalous release on account of, for example, swelling, while values below this indicated sub-diffusive release. Fitting of the release curves for both static release assays yielded diffusional exponents of n˜0.39 for the capillary setup and n˜0.66 for dialysis setup. These results may indicate that excess buffer and fluid motion (e.g., observed in the floating dialysis cassette), which does not fully recapitulate the gelatinous mixture of hyaluronic acid and other biopolymers, proteins, and cells of the VH, leads to anomalous release in the dialysis tube, whereas sub-diffusive release is observed when erosion is limited in the capillaries.

To evaluate the release of bimatoprost from the PNP hydrogels in a dynamic assay, an Agilent 400-DS Apparatus 7 instrument was utilized, which is often used for dissolution testing, batch analysis, and quality control of drug-eluting stents and medicated contact lenses, among other drug delivery technologies. Dynamic release was performed using the 400-DS at 37° C. in a 10 mL cell. Each reciprocating sample holder (n=2) of the 400-DS comprised a mesh basket and contained 100 μL of PNP hydrogel with bimatoprost (0.74 mg mL⁻¹) immersed in the dissolution media (5 mL PBS). The mesh basket and contents were moved up and down with a programmed dipping rate of one dipping cycle per minute (1 DPM). At each sampling point, 0.1 mL of the medium was withdrawn through the auto-sampling port into an HPLC vial. The dissolution duration was 72 hours and samples were taken at: 0.5, 4, 8, 24, 36, 48, 60, and 72 hours. Release profiles for bimatoprost and hydrogel component HPMC-C₁₂ and PEG-PLA nanoparticles were quantified using HPLC-CAD as described in supplemental methods. Data were fit with a one phase-decay in GraphPad Prism and the half-life of release was determined. Ritger-Peppas analysis

$$\frac{{M}_{t}}{{M}_{inf}} = {kt}^{n}$$

was performed by fitting data with power law in GraphPad Prism to find k and n.

As shown in FIG. 10D, the release half-life in this dynamic assay was determined to be only 0.1 day, significantly shorter than the release half-lives observed in either static release assay due to the additional shearing forces that lead to the rapid and complete dissolution of the PNP hydrogels. Indeed, the PEG-PLA NP and HPMC-C₁₂ components of the hydrogel were quantified in the releasate and showed similar release profiles as the entrapped bimatoprost. This observation suggests that release of the cargo from these hydrogels is driven primarily by dissolution-based erosion of the hydrogels and corroborates the sub-diffusive release observed in the static capillary assays described above. These results demonstrate that PNP hydrogels are physically crosslinked and completely dissolve away on a similar timescale as the release of the encapsulated drug. This feature is advantageous as it potentially eliminates the amount of excess material remaining following cargo release, which is a beneficial attribute for LAD technologies used in the eye. Comparison of these three assays highlights the importance of considering the final application of an LAD material in vivo when selecting a configuration setup for cargo release assays.

### Example 5: Intravitreal Administration of PNP Hydrogel in Rabbits

This example describes in vivo characterization of bimatoprost release from a PNP-2-10 hydrogel prepared as described in Example 1. The PNP hydrogel was characterized in vivo in New Zealand white (NZW) rabbits, a well-established preclinical model for investigating the safety of ocular therapeutic candidates.

All animal procedures were conducted in accordance and compliance with internal IACUC approved standards and the ARVO Statement for the Use of Animals in Ophthalmic and Vision Research. Eight male NZW rabbits (˜6 months old) received a single bilateral dose of 50 μL PNP hydrogel alone or with bimatoprost (0.16 mg mL⁻¹) by ITV injection.

Rabbits were placed under general anesthesia by an intramuscular (IM) cocktail of 7 mg kg⁻¹ ketamine HCl, 0.005 mg kg⁻¹ dexmedetomidine HCl, and 0.1 mg kg⁻¹ hydromorphone. Once appropriately sedated, a maintenance dose of isoflurane was delivered through a V-gel tube. To induce pupil dilation, 1% tropicamide was applied to both eyes prior to dosing procedure. A wire speculum was inserted. The eyelid margins and conjunctiva overlying the injection site were sterilized with 5% ophthalmic povidone iodine solution and 0.5% proparacaine hydrochloride was administered as a topical anesthetic. PNP hydrogel was administered via ITV injection into the inferotemporal quadrant of the globe using a 31-gauge×8 mm needle, affixed to an insulin syringe. Upon completion of hydrogel administration to both eyes, isoflurane administration was halted, v-gel tube removed, and atipamezole administered for reversal of dexmedetomidine HCl.

For animal dosing, hydrogel material (˜200 μL) with or without bimatoprost (0.16 mg mL⁻¹) was stored at 4° C. in sterile syringes enclosed in sterile 50 mL falcon tubes (one per dose) until the day of dosing. On the day of dosing, >50 μL of material was back-loaded into individual dosing syringes (0.3 mL insulin syringe, 31-gauge×8 mm needle, one per eye) and the syringe plunger was used to slowly concentrate the gel at the tip of the needle, careful to avoid bubbles in the hydrogel. μ50 L was injected intravitreally per eye.

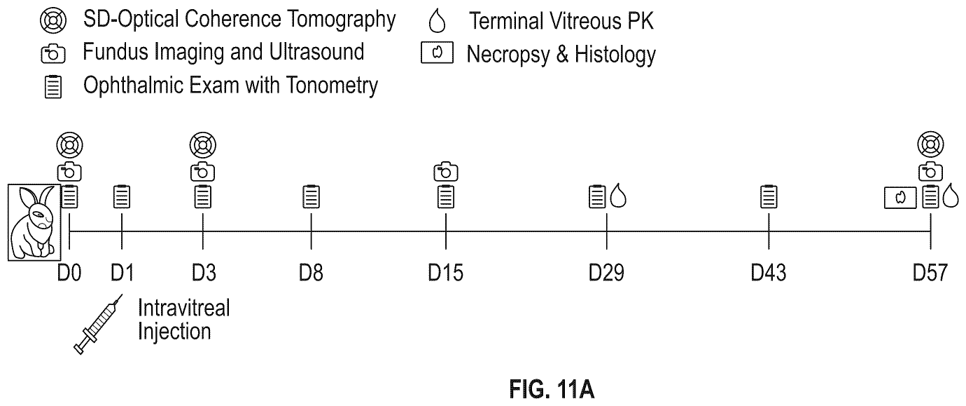

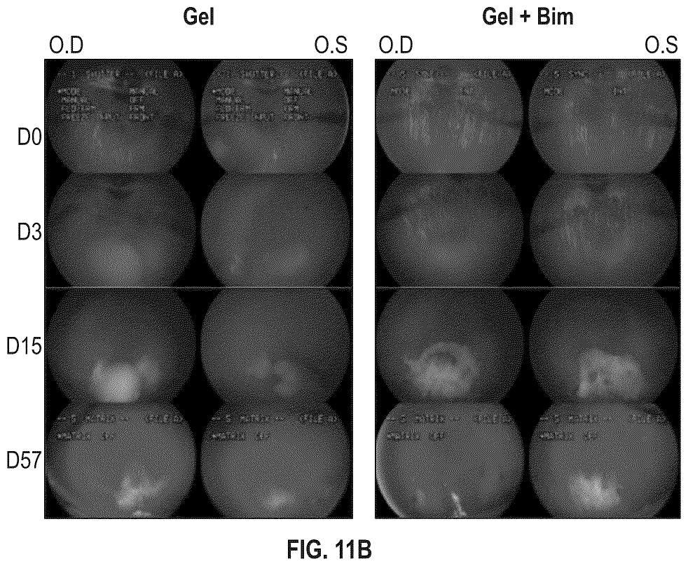

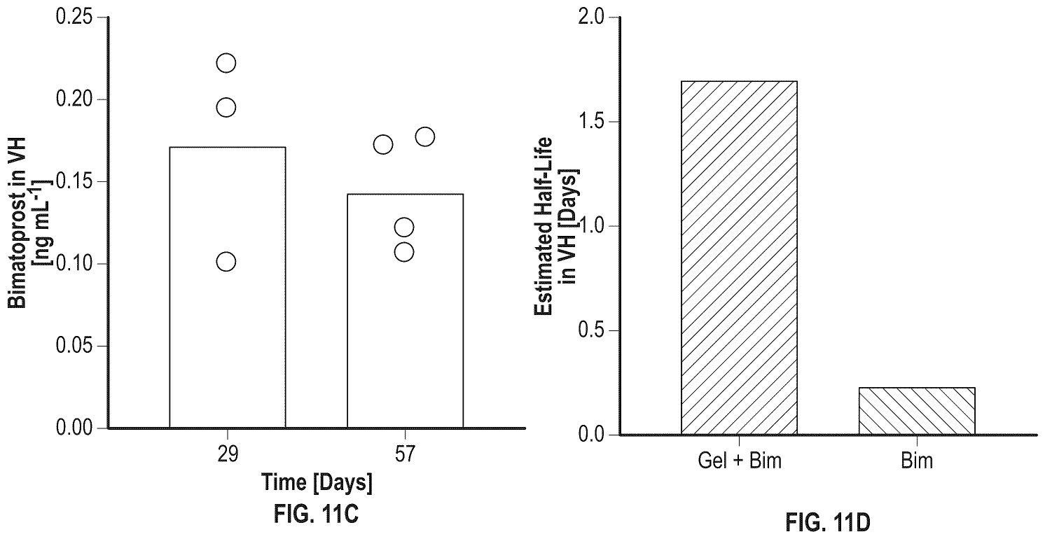

For these in vivo studies, and as detailed above, a bimatoprost dose of 8 μg and an injection volume of 50 μL per eye were targeted to minimize any potential adverse effects due to the drug cargo or administration protocol and to match the dosing used for in vitro characterization. Bilateral ITV injections delivered PNP hydrogels without bimatoprost (Gel) to two rabbits (four eyes total), and PNP hydrogels with bimatoprost (Gel+Bim) to six rabbits (twelve eyes total). Both formulations were confirmed to be endotoxin-free prior to administration. Rabbits were monitored for two months following ITV injection to assess ocular tolerability by OE comprised of slit-lamp biomicroscopy, indirect ophthalmoscopy, and rebound tonometry for IOP measurements. Wide-field color fundus and ultrasound imaging were used to monitor the appearance of the hydrogel and spectral domain-optical coherence tomography (SD-OCT) was used to qualitatively assess hydrogel-related changes to retinal layer thickness. Following euthanasia, ocular tissues were collected for pharmacokinetic (PK) analysis of bimatoprost in VH and histopathological analysis to evaluate any microscopic changes. A timeline of rabbit tolerability and PK study is shown in FIG. 11A.

Ophthalmic examinations, including slit-lamp biomicroscopy, indirect ophthalmoscopy, and rebound tonometry, were conducted to assess any intraocular changes and to characterize the hydrogel for up to eight weeks following ITV injection. Spectral Domain Optical Coherence Tomography (SD-OCT) imaging occurred sequentially on sedated rabbits using a Heidelberg Engineering Spectralis with attached 30-degree lens to document any retinal changes. SD-OCT imaging captured several scans including a radially oriented line scan running underneath the injected PNP hydrogel. Wide-field color fundus and VH imaging (RetCam 3 with attached 130-degree lens) and B-scan ultrasonography (Philips Epiq 7G) documented the visibility and position of the injected PNP hydrogel.

Immediately following administration, the hydrogels were visualized as a smooth, faintly opaque depot in the inferior VH. Few instances of spherical foci, presumed to be air bubbles, were observed at the surface or within the depot in both the Gel and Gel+Bim groups. By day 3, an increase in spherical foci were visualized within the hydrogel in both groups and the edges of the hydrogel depots appeared discontinuous. As the study progressed, the hydrogel depots were subjectively less smooth and defined, with strand-like edges and refractive precipitates or cell-sized particles visible near or within the depot. These precipitates and particles were presumed to be components of the hydrogel undergoing degradation and dissolution and were more numerous in eyes dosed with Gel+Bim. Fundus photographs taken at regular intervals (see FIG. 11B) confirmed the hydrogel depot was diminishing in size and definition over time, indicating degradation and dissolution of the hydrogel depots.

At days 29 and 57, two rabbits in the Gel+Bim group were euthanized (n=4 eyes per timepoint) and VH was collected and analyzed by LC-MS to measure bimatoprost concentration. As shown in FIG. 11C, detectable levels of bimatoprost were found even at day 57, indicating the PNP hydrogel was successful at sustaining the release of this small molecule. With reference to FIG. 11D, it was estimated that the effective elimination half-life of bimatoprost from the eye was 1.7 days using the initial dose of 8 μg in a standard volume range for rabbit VH (1.15-1.7 mL). This estimated half-life falls within the range of half-lives observed from the in vitro static release assays discussed above. As the elimination half-life of bimatoprost following IV administration is reported to be only 45 minutes, the effective half-life measured here for the PNP hydrogel-based formulations in VH represents an almost two-order-of-magnitude improvement. A pharmacokinetic model built on experimental rabbit data and physiochemical properties of various drugs (molecular weight and lipophilicity) was used to estimate the half-life of bimatoprost in VH. When applied to bimatoprost, the model conservatively predicted a half-life of 0.225 days, in agreement with known elimination half-lives of other small molecules in VH. Therefore, the PNP hydrogel increased the effective elimination half-life of bimatoprost in VH by more than 7.5-fold from 0.225 days to 1.7 days. These initial data demonstrate measurable bimatoprost in VH for almost two months.

Following euthanasia, eyes were enucleated and dissected. For eyes designated for histopathology, the globe with the optic nerve was fixed in modified Davidson's fixative for approximately 48 hours, then transferred to 70% ethanol for histologic evaluation. Histologic evaluation was completed by StageBio (Mason, Ohio). An axial superoinferior section was taken to include the optic nerve head and lens, with 3 step sections taken at 100 μm intervals. The temporal and nasal calottes were placed axial-side down and 3 step sections were taken at 100 μm each following embedding in paraffin. Sectioned blocks were stained with hematoxylin and eosin (H&E). H&E stained tissues were evaluated by an ACVP-board certified veterinary pathologist.

For eyes designated for PK analysis, VH was harvested and frozen at −70° C. Prior to analysis, material was thawed and 100 μL of each VH sample was pipetted into individual cluster tubes, vortexed for 10 minutes, and centrifuged at 3700 rpm for 10 minutes at 4° C. After centrifugation, supernatant was directly injected onto the autosampler for LC-MS/MS detection as described below.

A Nexera UPLC system (Shimadzu, Kyoto, Japan) with a Phenomenex XB-C18 column (50×2.1 mm, 2.7 μm) was used to analyze bimatoprost in VH. The gradient elution was 0.1% formic acid in water and 0.1% formic acid in acetonitrile with flow rate 1.1 mL min⁻¹. A QTrap® 5500 tandem mass spectrometer (Sciex, Foster City, CA) with Turboionspray (TIS) interface was operated in positive ionization mode with multiple reaction monitoring (MRM) for LC-MS/MS analysis. Bimatoprost standard was purchased from BioVision Inc and prepared at 1 mg mL⁻¹ in 100% DMSO for the stock solution and serially diluted for a calibration curve from 2000 ng mL⁻¹ to 0.1 ng mL⁻¹.

### Example 6: In Vivo Tolerability Evaluation

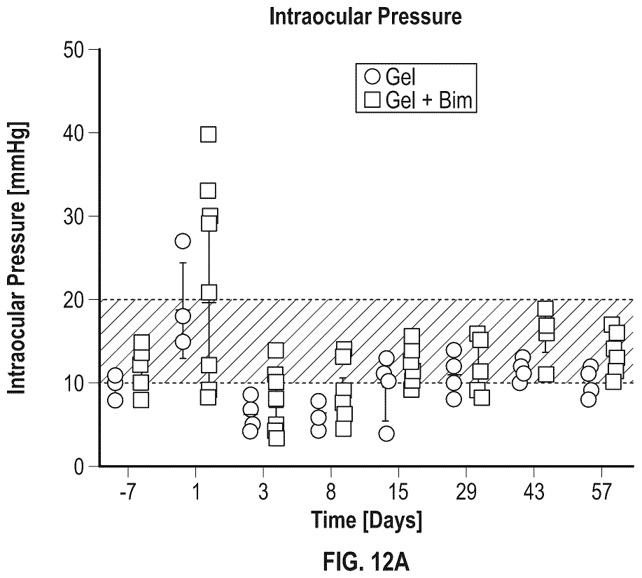

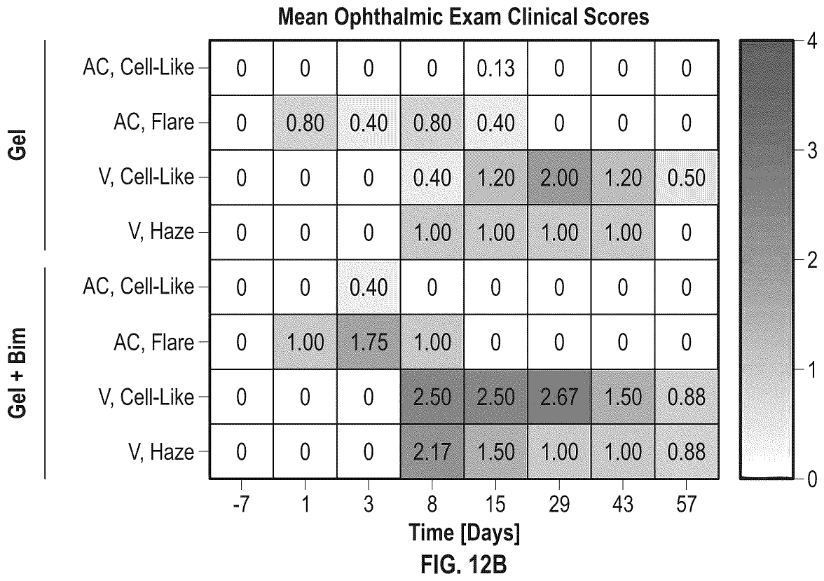

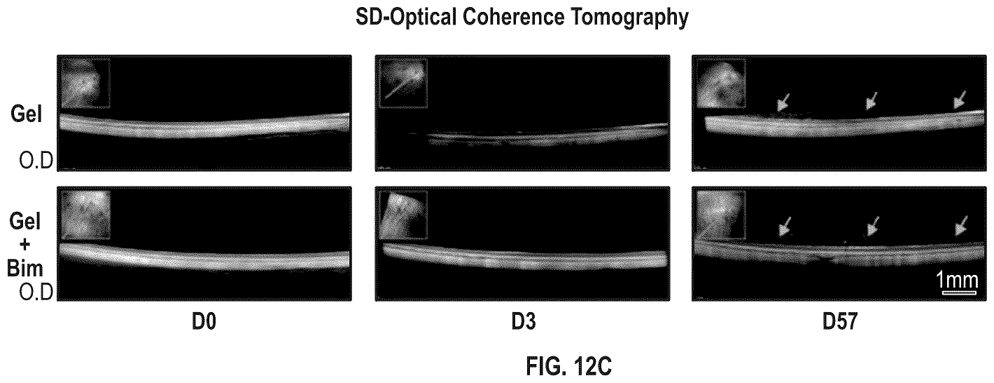

This example describes an analysis of the tolerability of the PNP-2-10 hydrogel in the environment of the eye. A useful parameter for any long-term drug delivery vehicle is tolerability in the sensitive and immune-privileged tissues of the eye. Possible adverse effects of the PNP hydrogels were evaluated through several in-life measurements including OE with tonometry, fundus and ultrasound imaging, and SD-OCT. IOP was transiently elevated above the normal range (10-20 mmHg) for both groups (Gel and Gel+Bim) immediately following administration, which is expected for ITV injection (FIG. 12A). In subsequent intervals, IOP values were found to be below normal, and trace aqueous flare was observed, consistent with ITV injection-related breakdown of the blood-ocular barrier (BOB). While the therapeutic effect of bimatoprost is decreased IOP, a sustained drop in IOP was not expected in these studies as a disease model exhibiting elevated IOP was not being used. As the BOB integrity returned, IOP values returned to normal levels and aqueous flare resolved in a majority of eyes in both groups, and both metrics remained within a normal range for the duration of the study. Generally, these IOP findings were more pronounced in eyes dosed with Gel+Bim, presumably on account of bimatoprost's known impact on BOB integrity that may exacerbate preexisting inflammation.

As the IOP and aqueous flare associated with injection and bimatoprost subsided, a minor inflammatory reaction was observed in the vitreous. This reaction was characterized by trace to moderate haze and refractive, cell-like material that was likely a mix of inflammatory cells and hydrogel degradant products, suggestive of a foreign body response to the PNP hydrogel (FIG. 12B). Qualitatively, the measure of cell-like material peaked at day 29 and resolved by the terminal time point, with slightly greater severity maintained in eyes dosed with Gel+Bim. SD-OCT and ultrasound did not reveal any alterations to the retina, but an accumulation of hyperreflective debris within the VH immediately adjacent to the retinal surface, presumed to be hydrogel degradant products, was apparent in both groups at study termination (FIG. 12C).

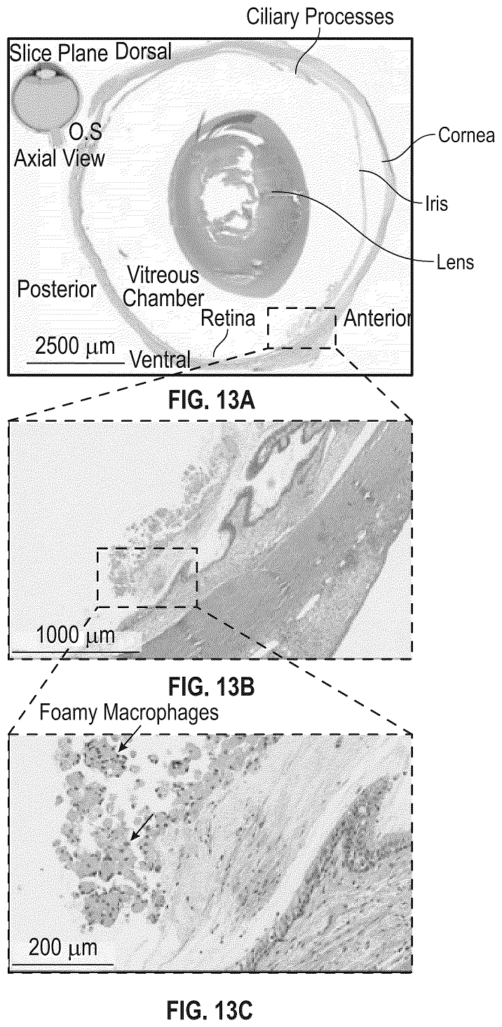

Microscopic examination of all eyes was conducted at study termination to identify any changes related to the ITV injected PNP hydrogel. Slides were prepared from paraffin-embedded eyes and stained with hematoxylin and eosin (H&E). Minimal to mild infiltration and accumulation of foamy macrophages and multinucleated giant cells in the VH were observed in eyes from both groups (FIGS. 13A-13C). The cells were present in clusters anteriorly and inferiorly near the ciliary body (region of hydrogel deposition), scattered in smaller clusters extending inferiorly along the retina toward the back of the eye, and in the iridocorneal drainage angle, likely in the process of clearing from the eye. These cells were also observed in close association with minimal, patchy fibroplasia. The presence of macrophages, and particularly multinucleated giant cells, suggests a mild foreign body response to the PNP hydrogel. Injection procedure-related minimal to moderate mixed cell inflammation of the bulbar conjunctiva and/or sclera was also observed.

Foreign body responses have been observed in rabbits following ITV injection of other materials, with foreign body response-associated inflammation observable during OE soon following injection or implantation. One contributing factor to the observed foreign body response to the PNP hydrogels may be the difference in mechanical properties between the hydrogels and VH. While the PNP hydrogels exhibit low moduli, their storage modulus of ˜150 Pa is still higher than the modulus of VH, which has been shown to be between 1 and 10 Pa. In contrast to other LAD technologies in rabbits, OEs captured only a mild inflammatory reaction that subsided over time in this study. Furthermore, no major retinal changes were observed upon OCT or ultrasound. Microscopic findings of fibroplasia in response to the presence of PNP hydrogels were observed. Without being bound by theory, it is hypothesized that this observed mild foreign body response could be reduced or eliminated by improved handling and/or decontamination protocols, reducing the number of carbon atoms in the lipid tails of the polymer (as described above), changing the composition of the degradable polymer block in the nanoparticles (such as using a PEG-alternative and/or a PLA alternative), changing the cellulose polymer backbone, and others. The cellulose polymer backbone may comprise, for example, other cellulosic polymers such as methylcellulose or carboxymethylcellulose. In some embodiments, the hydrogel polymer could comprise HA.

### ADDITIONAL EXAMPLES

Additional examples of aspects of the present technology are described below as numbered clauses (1, 2, 3, etc.) for convenience. These are provided as examples and do not limit the subject technology.

Clause 1. A composition for treating a disease or condition, the composition comprising:
a dynamic hydrogel comprising a polymer and a plurality of nanoparticles, wherein the polymer is non-covalently crosslinked with the plurality of nanoparticles; and
an ocular therapeutic encapsulated by the dynamic hydrogel.

Clause 2. The composition of Clause 1, wherein the ocular therapeutic comprises a prostaglandin analog (PGA).

Clause 3. The composition of Clause 2, wherein the ocular therapeutic is bimatoprost.

Clause 4. The composition of any one of Clauses 1 to 3, wherein the ocular therapeutic is encapsulated in the dynamic hydrogel via hydrophobic interactions between the ocular therapeutic and the dynamic hydrogel.

Clause 5. The composition of any one of Clauses 1 to 4, wherein the polymer comprises a hydrophobically-modified polysaccharide.

Clause 6. The composition of Clause 5, wherein the hydrophobically-modified polysaccharide comprises a hydrophobically-modified cellulose derivative.

Clause 7. The composition of Clause 6, wherein the hydrophobically-modified cellulose derivative is dodecyl-modified hydroxypropylmethylcellulose (HPMC-C₁₂).

Clause 8. The composition of any one of Clauses 1 to 7, wherein the plurality of nanoparticles comprises a plurality of polymeric nanoparticles.

Clause 9. The composition of Clause 8, wherein the plurality of polymeric nanoparticles comprises a plurality of amphiphilic nanoparticles.

Clause 10. The composition of Clause 8 or Clause 9, wherein the plurality of polymeric nanoparticles comprises a plurality of poly(ethylene glycol)-block-poly(lactic acid) (PEG-PLA) nanoparticles.

Clause 11. The composition of any one of Clauses 1 to 10, wherein a concentration of the polymer in the dynamic hydrogel is within a range from 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %.

Clause 12. The composition of any one of Clauses 1 to 11, wherein a concentration of the nanoparticles in the dynamic hydrogel is within a range from 1 wt % to 12 wt %, 1 wt % to 10 wt %, 1 wt % to 8 wt %, 1 wt % to 5 wt %, 1 wt % to 3 wt %, 3 wt % to 12 wt %, 3 wt % to 10 wt %, 3 wt % to 8 wt %, 3 wt % to 5 wt %, 5 wt % to 12 wt %, 5 wt % to 10 wt %, 5 wt % to 8 wt %, 8 wt % to 12 wt %, 8 wt % to 10 wt %, or 10 wt % to 12 wt %.

Clause 13. The composition of any one of Clauses 1 to 12, wherein the dynamic hydrogel encapsulating the ocular therapeutic has a storage modulus within a range from 10 Pa to 1000 Pa, or 50 Pa to 500 Pa when measured at 25° C. over an angular frequency of 0.1 rad/s to 100 rad/s within a linear viscoelastic region of the dynamic hydrogel.

Clause 14. The composition of any one of Clauses 1 to 13, wherein the dynamic hydrogel encapsulating the ocular therapeutic has a yield stress within a range from 1 Pa to 500 Pa, or 20 Pa to 200 Pa when measured at 25° C.

Clause 15. The composition of any one of Clauses 1 to 14, wherein the dynamic hydrogel encapsulating the ocular therapeutic has a viscosity within a range from 100 mPa-s to 1000 mPa-s when measured at 25° C. at a shear rate of 1000 s⁻¹.

Clause 16. The composition of any one of Clauses 1 to 15, wherein, upon administration to a subject, the composition delivers the ocular therapeutic to the subject over a treatment period of at least 7 days, 8 days, 9 days, 10 days, 11 days, 12 days, 13 days, 14 days, 28 days, 35 days, 40 days, 50 days, 60 days, 70 days, 80 days, 90 days, 100 days, 110 days, 120 days, 150 days, 180 days, 6 months, 7 months, 8 months, 9 months, 10 months, 11 months, 12 months, or 1 year.

Clause 17. A method of treating a disease or condition, the method comprising: administering a composition to a subject, wherein the composition comprises:
a dynamic hydrogel comprising a polymer and a plurality of nanoparticles, wherein the polymer is non-covalently crosslinked with the plurality of nanoparticles, and
an ocular therapeutic encapsulated by the dynamic hydrogel.

Clause 18. The method of Clause 17, wherein the ocular therapeutic comprises a PGA.

Clause 19. The method of Clause 18, wherein the ocular therapeutic is bimatoprost.

Clause 20. The method of any one of Clauses 17 to 19, wherein the ocular therapeutic is encapsulated in the dynamic hydrogel via hydrophobic interactions between the ocular therapeutic and the dynamic hydrogel.

Clause 21. The method of any one of Clauses 17 to 20, wherein the polymer comprises a hydrophobically-modified polysaccharide.

Clause 22. The method of Clause 21, wherein the hydrophobically-modified polysaccharide comprises a hydrophobically-modified cellulose derivative.

Clause 23. The method of Clause 22, wherein the hydrophobically-modified cellulose derivative is dodecyl-modified hydroxypropylmethylcellulose (HPMC-C₁₂).

Clause 24. The method of any one of Clauses 17 to 23, wherein the plurality of nanoparticles comprises a plurality of polymeric nanoparticles.

Clause 25. The method of Clause 24, wherein the plurality of polymeric nanoparticles comprises a plurality of amphiphilic nanoparticles.

Clause 26. The method of Clause 24 or Clause 25, wherein the plurality of polymeric nanoparticles comprises a plurality of poly(ethylene glycol)-block-poly(lactic acid) (PEG-PLA) nanoparticles.

Clause 27. The method of any one of Clauses 17 to 26, wherein a concentration of the polymer in the dynamic hydrogel is within a range from 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %.

Clause 28. The method of any one of Clauses 17 to 27, wherein a concentration of the nanoparticles in the dynamic hydrogel is within a range from 1 wt % to 12 wt %, 1 wt % to 10 wt %, 1 wt % to 8 wt %, 1 wt % to 5 wt %, 1 wt % to 3 wt %, 3 wt % to 12 wt %, 3 wt % to 10 wt %, 3 wt % to 8 wt %, 3 wt % to 5 wt %, 5 wt % to 12 wt %, 5 wt % to 10 wt %, 5 wt % to 8 wt %, 8 wt % to 12 wt %, 8 wt % to 10 wt %, or 10 wt % to 12 wt %.

Clause 29. The method of any one of Clauses 17 to 28, wherein the dynamic hydrogel encapsulating the ocular therapeutic has a storage modulus within a range from 10 Pa to 1000 Pa, or 50 Pa to 500 Pa when measured at 25° C. over an angular frequency of 0.1 rad/s to 100 rad/s within a linear viscoelastic region of the dynamic hydrogel.

Clause 30. The method of any one of Clauses 17 to 29, wherein the dynamic hydrogel encapsulating the ocular therapeutic has a yield stress within a range from 1 Pa to 500 Pa, or 20 Pa to 200 Pa when measured at 25° C.

Clause 31. The method of any one of Clauses 17 to 30, wherein the dynamic hydrogel encapsulating the ocular therapeutic has a viscosity within a range from 100 mPa-s to 1000 mPa-s when measured at 25° C. at a shear rate of 1000 s⁻¹.

Clause 32. The method of any one of Clauses 17 to 31, wherein, upon administration to a subject, the composition delivers the ocular therapeutic to the subject over a treatment period of at least 7 days, 8 days, 9 days, 10 days, 11 days, 12 days, 13 days, 14 days, 28 days, 35 days, 40 days, 50 days, 60 days, 70 days, 80 days, 90 days, 100 days, 110 days, 120 days, 150 days, 180 days, 6 months, 7 months, 8 months, 9 months, 10 months, 11 months, 12 months, or 1 year.

### CONCLUSION

Although many of the embodiments are described above with respect to systems, devices, and methods for localized, sustained release of ocular therapeutics, the technology is applicable to other applications and/or other approaches, such as delivery of other therapeutic agents to the eye. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-13C.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art. The terms “about” and “approximately,” in reference to a number, are used herein to include numbers that fall within a range of 10%, 5%, or 1% in either direction (greater than or less than) the number unless otherwise stated or otherwise evident from the context (except where such number would exceed 100% of a possible value).

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

As used herein, the term “subject” may refer to any animal, including but not limited to, humans and non-human animals (e.g., dogs, cats, cows, horses, sheep, pigs, poultry, fish, crustaceans, etc.).

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A composition for treating a disease or condition, the composition comprising:
a dynamic hydrogel comprising a polymer and a plurality of nanoparticles, wherein the polymer is non-covalently crosslinked with the plurality of nanoparticles; and
an ocular therapeutic encapsulated by the dynamic hydrogel.

2. The composition of claim 1, wherein the ocular therapeutic comprises a prostaglandin analog (PGA).

3. The composition of claim 2, wherein the ocular therapeutic is bimatoprost.

4. The composition of any one of claims 1 to 3, wherein the ocular therapeutic is encapsulated in the dynamic hydrogel via hydrophobic interactions between the ocular therapeutic and the dynamic hydrogel.

5. The composition of any one of claims 1 to 4, wherein the polymer comprises a hydrophobically-modified polysaccharide.

6. The composition of claim 5, wherein the hydrophobically-modified polysaccharide comprises a hydrophobically-modified cellulose derivative.

7. The composition of claim 6, wherein the hydrophobically-modified cellulose derivative is dodecyl-modified hydroxypropylmethylcellulose (HPMC-C₁₂).

8. The composition of any one of claims 1 to 7, wherein the plurality of nanoparticles comprises a plurality of polymeric nanoparticles.

9. The composition of claim 8, wherein the plurality of polymeric nanoparticles comprises a plurality of amphiphilic nanoparticles.

10. The composition of claim 8 or claim 9, wherein the plurality of polymeric nanoparticles comprises a plurality of poly(ethylene glycol)-block-poly(lactic acid) (PEG-PLA) nanoparticles.

11. The composition of any one of claims 1 to 10, wherein a concentration of the polymer in the dynamic hydrogel is within a range from 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %.

12. The composition of any one of claims 1 to 11, wherein a concentration of the nanoparticles in the dynamic hydrogel is within a range from 1 wt % to 12 wt %, 1 wt % to 10 wt %, 1 wt % to 8 wt %, 1 wt % to 5 wt %, 1 wt % to 3 wt %, 3 wt % to 12 wt %, 3 wt % to 10 wt %, 3 wt % to 8 wt %, 3 wt % to 5 wt %, 5 wt % to 12 wt %, 5 wt % to 10 wt %, 5 wt % to 8 wt %, 8 wt % to 12 wt %, 8 wt % to 10 wt %, or 10 wt % to 12 wt %.

13. The composition of any one of claims 1 to 12, wherein the dynamic hydrogel encapsulating the ocular therapeutic has a storage modulus within a range from 10 Pa to 1000 Pa, or 50 Pa to 500 Pa when measured at 25° C. over an angular frequency of 0.1 rad/s to 100 rad/s within a linear viscoelastic region of the dynamic hydrogel.

14. The composition of any one of claims 1 to 13, wherein the dynamic hydrogel encapsulating the ocular therapeutic has a yield stress within a range from 1 Pa to 500 Pa, or 20 Pa to 200 Pa when measured at 25° C.

15. The composition of any one of claims 1 to 14, wherein the dynamic hydrogel encapsulating the ocular therapeutic has a viscosity within a range from 100 mPa-s to 1000 mPa-s when measured at 25° C. at a shear rate of 1000 s⁻¹.

16. The composition of any one of claims 1 to 15, wherein, upon administration to a subject, the composition delivers the ocular therapeutic to the subject over a treatment period of at least 7 days, 8 days, 9 days, 10 days, 11 days, 12 days, 13 days, 14 days, 28 days, 35 days, 40 days, 50 days, 60 days, 70 days, 80 days, 90 days, 100 days, 110 days, 120 days, 150 days, 180 days, 6 months, 7 months, 8 months, 9 months, 10 months, 11 months, 12 months, or 1 year.

17. A method of treating a disease or condition, the method comprising:
administering a composition to a subject, wherein the composition comprises:
a dynamic hydrogel comprising a polymer and a plurality of nanoparticles, wherein the polymer is non-covalently crosslinked with the plurality of nanoparticles, and
an ocular therapeutic encapsulated by the dynamic hydrogel.

18. The method of claim 17, wherein the ocular therapeutic comprises a PGA.

19. The method of claim 18, wherein the ocular therapeutic is bimatoprost.

20. The method of any one of claims 17 to 19, wherein the ocular therapeutic is encapsulated in the dynamic hydrogel via hydrophobic interactions between the ocular therapeutic and the dynamic hydrogel.

21. The method of any one of claims 17 to 20, wherein the polymer comprises a hydrophobically-modified polysaccharide.

22. The method of claim 21, wherein the hydrophobically-modified polysaccharide comprises a hydrophobically-modified cellulose derivative.

23. The method of claim 22, wherein the hydrophobically-modified cellulose derivative is dodecyl-modified hydroxypropylmethylcellulose (HPMC-C₁₂).

24. The method of any one of claims 17 to 23, wherein the plurality of nanoparticles comprises a plurality of polymeric nanoparticles.

25. The method of claim 24, wherein the plurality of polymeric nanoparticles comprises a plurality of amphiphilic nanoparticles.

26. The method of claim 24 or claim 25, wherein the plurality of polymeric nanoparticles comprises a plurality of poly(ethylene glycol)-block-poly(lactic acid) (PEG-PLA) nanoparticles.

27. The method of any one of claims 17 to 26, wherein a concentration of the polymer in the dynamic hydrogel is within a range from 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %.

28. The method of any one of claims 17 to 27, wherein a concentration of the nanoparticles in the dynamic hydrogel is within a range from 1 wt % to 12 wt %, 1 wt % to 10 wt %, 1 wt % to 8 wt %, 1 wt % to 5 wt %, 1 wt % to 3 wt %, 3 wt % to 12 wt %, 3 wt % to 10 wt %, 3 wt % to 8 wt %, 3 wt % to 5 wt %, 5 wt % to 12 wt %, 5 wt % to 10 wt %, 5 wt % to 8 wt %, 8 wt % to 12 wt %, 8 wt % to 10 wt %, or 10 wt % to 12 wt %.

29. The method of any one of claims 17 to 28, wherein the dynamic hydrogel encapsulating the ocular therapeutic has a storage modulus within a range from 10 Pa to 1000 Pa, or 50 Pa to 500 Pa when measured at 25° C. over an angular frequency of 0.1 rad/s to 100 rad/s within a linear viscoelastic region of the dynamic hydrogel.

30. The method of any one of claims 17 to 29, wherein the dynamic hydrogel encapsulating the ocular therapeutic has a yield stress within a range from 1 Pa to 500 Pa, or 20 Pa to 200 Pa when measured at 25° C.

31. The method of any one of claims 17 to 30, wherein the dynamic hydrogel encapsulating the ocular therapeutic has a viscosity within a range from 100 mPa-s to 1000 mPa-s when measured at 25° C. at a shear rate of 1000 s⁻¹.

32. The method of any one of claims 17 to 31, wherein, upon administration to a subject, the composition delivers the ocular therapeutic to the subject over a treatment period of at least 7 days, 8 days, 9 days, 10 days, 11 days, 12 days, 13 days, 14 days, 28 days, 35 days, 40 days, 50 days, 60 days, 70 days, 80 days, 90 days, 100 days, 110 days, 120 days, 150 days, 180 days, 6 months, 7 months, 8 months, 9 months, 10 months, 11 months, 12 months, or 1 year.
